(19) United States
(12) Patent Application Publication
(10) Pub. No.: US 2018/0035898 A1
(43) Pub. Date: Feb. 8, 2018

(54) CHANGE IN PHYSIOLOGICAL PARAMETER IN RESPONSE TO EXERTION EVENT

(71) Applicant: Medtronic, Inc., Minneapolis, MN (US)

(72) Inventor: Bruce D. GUNDERSON, Plymouth, MN (US)

(21) Appl. No.: 15/604,044

(22) Filed: May 24, 2017

Related U.S. Application Data
(60) Provisional application No. 62/370,113, filed on Aug. 2, 2016.

(51) Int. Cl. A61B 5/0205; A61B 5/00

(52) U.S. Cl. CPC A61B 5/0205; A61B 5/746; A61B 5/686; A61B 2562/0219; A61B 5/1116

(57) ABSTRACT

A method for monitoring health of a subject based on a physiological response to physical exertion, by processing circuitry of a medical device system, is described that includes detecting a plurality of exertion events of the subject based on a first sensed signal that varies as a function of movement of the subject. The method further includes determining a response of a physiological parameter of the subject to the exertion event for each of the detected exertion events based on second sensed signal that varies as a function of the physiological parameter. The method further includes determining that a change in the responses over time crosses threshold and generating an alert to a user based on the determination that the change crosses the threshold.

### RELATED APPLICATIONS

This application claims the benefit of U.S. Provisional Application No. 62/370,113, filed Aug. 2, 2016 and is incorporated by reference herein.

### FIELD

The disclosure relates to methods and systems for measuring physiological parameters, such as heart rate.

### BACKGROUND

Implantable medical devices (IMDs) and external, e.g., wearable, medical devices, including implantable pacemakers and implantable cardioverter-defibrillators (ICDs), record cardiac electrogram (EGM) signals for sensing cardiac events, e.g., P-waves and R-waves. IMDs detect episodes of bradycardia, tachycardia and/or fibrillation from the sensed cardiac events, and respond to the episodes as needed with pacing therapy or high-voltage anti-tachyarrhythmia shocks, e.g., cardioversion or defibrillation shocks. Some IMDs include, or are or part of a system that includes, sensors that generate other physiological signals, such as signals that vary based on patient movement or activity, cardiovascular pressure, blood oxygen saturation, edema, or thoracic impedance.

### SUMMARY

In general, this disclosure is directed to techniques for monitoring physiological responses to physical exertion. The example techniques include monitoring first sensed signals that vary as a function of movement of the subject and second sensed signals that vary as a function of a physiological parameter. The example techniques include detecting exertion events based on the first sensed signals and determining a response of the physiological parameter to each exertion event based on the second sensed signals. The example techniques include determining that a change in the responses over time crosses, e.g., exceeds and/or falls below, a threshold or threshold range, and generating an alert to a user.

As one example, the disclosure is directed to a method for monitoring health of a subject based on a physiological response to physical exertion, by processing circuitry of a medical device system, that includes detecting a plurality of exertion events of the subject based on a first sensed signal that varies as a function of movement of the subject. The method further includes determining a response of a physiological parameter of the subject to the exertion event for each of the detected exertion events based on second sensed signal that varies as a function of the physiological parameter. The method further includes determining that a change in the responses over time crosses a threshold and generating an alert to a user based on the determination that the change crosses the threshold.

A medical device system configured to monitor health of a subject based on a physiological response to physical exertion comprising sensing circuitry configured to generate a first sensed signal that varies as a function of movement of the subject. The sensing circuitry is further configured to generate a second sensed signal that varies as a function of a physiological parameter of the subject. The medical device system further comprises processing circuitry configured to detect a plurality of exertion events of the subject based on the first sensed signal. The processing circuitry is further configured to, for each of the detected exertion events, determine a response of the physiological parameter of the subject to the exertion event based on the second sensed signal. The processing circuitry is further configured to determine that a change in the responses over time crosses a threshold. The processing circuitry is further configured to generate an alert to a user in response to the determination that the change crosses the threshold.

As another example, the disclosure is directed to a method for monitoring health of a subject based on a physiological response to physical exertion comprising, by processing circuitry of a medical device system, detecting a plurality of exertion events of the subject based on a first sensed signal that varies as a function of movement of the subject. The method further comprises, for each of the detected exertion events, determining a response of a physiological parameter of the subject to the exertion event based on second sensed signal that varies as a function of the physiological parameter. The method further comprises determining a trend in the responses over time crosses a threshold. The method further comprises generating an alert to a user based on the determination that the trend crosses the threshold.

In an additional example, the disclosure is directed to a non-transitory computer-readable storage medium comprising instructions, that when executed by processing circuitry of a medical device system, causes the medical device system to detect a plurality of exertion events of the subject based on a first sensed signal that varies as a function of movement of the subject. The instructions further cause the medical device system to determine a response of a physiological parameter of the subject to the exertion event for each of the detected exertion events based on second sensed signal that varies as a function of the physiological parameter. The instructions further cause the medical device system to determine that a change in the responses over time crosses a threshold and generate an alert to a user based on the determination that the change crosses the threshold.

In an additional example, the disclosure is directed to a medical device system comprising means for monitoring health of a subject based on a physiological response to physical exertion, by processing circuitry of a medical device system, that includes detecting a plurality of exertion events of the subject based on a first sensed signal that varies as a function of movement of the subject. The medical device system further comprises means for determining a response of a physiological parameter of the subject to the exertion event for each of the detected exertion events based on second sensed signal that varies as a function of the physiological parameter. The medical device system further comprises means for determining that a change in the responses over time crosses a threshold and means for generating an alert to a user based on the determination that the change crosses the threshold.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual drawing illustrating an example medical device system in conjunction with a patient.

FIG. 2 is a perspective drawing illustrating an example configuration of the implantable cardiac monitor of FIG. 1.

FIG. 3 is a conceptual drawing illustrating another example medical device system in conjunction with a patient.

FIGS. 4A-4C are front-view, side-view, and top-view conceptual drawings, respectively, illustrating another example medical device system in conjunction with a patient.

FIG. 5 is a conceptual drawing illustrating another example medical device system in conjunction with a patient.

FIG. 6 is a conceptual diagram illustrating an example configuration of the intracardiac pacing device of FIGS. 4A-5.

FIG. 7 is a functional block diagram illustrating an example configuration of an implantable medical device.

FIG. 8 is a functional block diagram illustrating an example configuration of an external device configured to communicate with one or more implantable medical devices.

FIG. 9 is a functional block diagram illustrating an example system that includes remote computing devices, such as a server and one or more other computing devices, that are connected to an implantable medical device and/or external device via a network.

FIG. 10 is a conceptual diagram illustrating sagittal, vertical and transverse axes in a three-dimensional coordinate system.

FIG. 11 is a timing diagram illustrating a chart of three accelerometer signals, where the three signals represent vertical acceleration, transverse acceleration, and sagittal acceleration, and a marker channel chart showing heart beats before, during, and after an exertion event.

FIG. 12 is a flowchart illustrating an example technique for determining whether a change in responses of a physiological parameter to an exertion event crosses a threshold, in accordance with this disclosure.

FIG. 13 is a flowchart illustrating an example technique for determining whether a change in heart-beat responses to a sit-stand transition crosses a specific threshold, in accordance with this disclosure.

The drawings and the description provided herein illustrate and describe various examples of the inventive methods, devices, and systems of the present disclosure. However, the methods, devices, and systems of the present disclosure are not limited to the specific examples as illustrated and described herein, and other examples and variations of the methods, devices, and systems of the present disclosure, as would be understood by one of ordinary skill in the art, are contemplated as being within the scope of the present application.

### DETAILED DESCRIPTION

In general, this disclosure describes example techniques related to monitoring the health of a subject based on physiological responses to physical exertion. To carry out the example techniques, a system may determine a response of a physiological parameter to exertion events, where the response may be an increase in heart rate, blood pressure, or respiration. The system may also determine that a change in the responses over time crosses a threshold. The change in the responses may indicate an abnormal or unhealthy condition in the subject. The system may generate an alert to inform a user of the possible abnormal or unhealthy condition, where the user may be the subject, a caretaker, a medical professional, an external device, or any other user. In the following description, references are made to illustrative examples. It is understood that other examples may be utilized without departing from the scope of the disclosure.

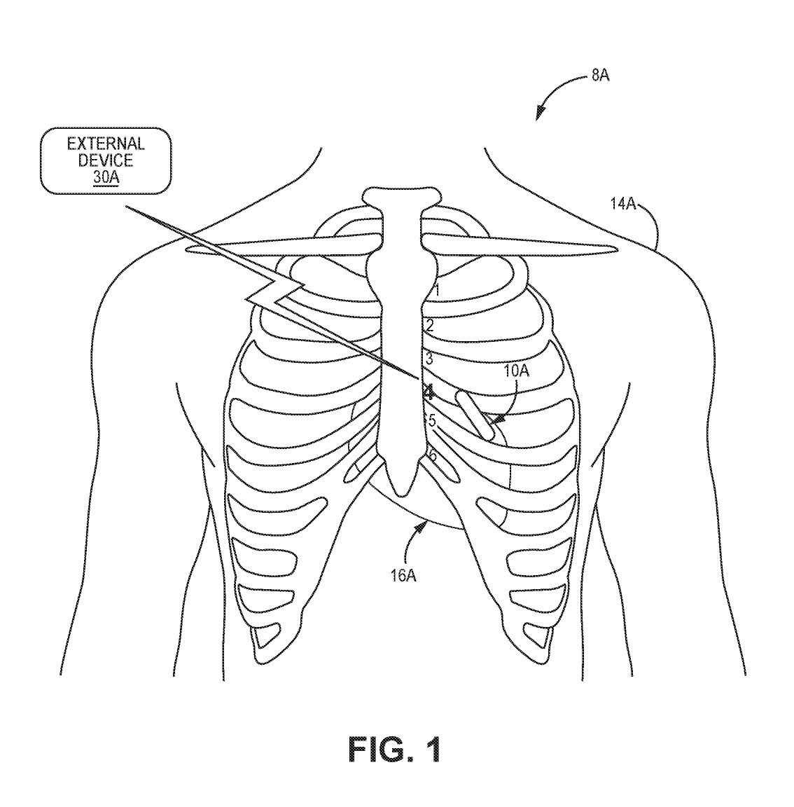

FIG. 1 is a conceptual drawing illustrating an example medical device system 8A in conjunction with a patient 14A. Medical device system 8A is an example of a medical device system configured to implement the techniques described herein for monitoring health of patient 14A based on the physiological response, such as heart rate, blood pressure, or respiration, to physical exertion, and responsively generating an alert in response to determining that a change in the physiological response over time crosses, e.g., exceeds and/or falls below, a threshold, e.g., threshold range. In the illustrated example, medical device system 8A includes an implantable medical device (IMD) 10A in communication with external device 30A.

In the illustrated example, IMD 10A is an insertable cardiac monitor (ICM) capable of sensing and recording cardiac EGM signals from a position outside of heart 16A, and will be referred to as ICM 10A hereafter. In some examples, ICM 10A includes or is coupled to one or more additional sensors that generate one or more physiological signals, such as signals that vary based on patient motion and/or posture, blood flow, or respiration. ICM 10A may monitor a physiological parameter such as heart rate, and ICM 10A may measure a change in the physiological parameter in response to an exertion event, such as a sit-stand transition detected based on a signal that varies as a function of patient motion or movement. ICM 10A may be implanted outside of the thorax of patient 14B, e.g., subcutaneously or submuscularly, such as the pectoral location illustrated in FIG. 1. In some examples, ICM 10B may take the form of a Reveal LINQ™ ICM, available from Medtronic plc, of Dublin, Ireland.

ICM 10A may transmit EGM signal data, cardiac rhythm episode data, and other physiological parameter data acquired by ICM 10A, to an external device 30A. For examples, ICM 10A may transmit any data described herein related to detection of exertion events, responses of one or more physiological parameters to the exertion events, changes in such responses over time, or alerts based on such changes to external device 30A. External device 30A may be a computing device, e.g., used in a home, ambulatory, clinic, or hospital setting, to communicate with ICD 10A via wireless telemetry. External device 30A may be coupled to a remote patient monitoring system, such as Carelink®, available from Medtronic plc, of Dublin, Ireland. External device 30A may be, as examples, a programmer, external monitor, or consumer device, e.g., smart phone.

External device 30A may be used to program commands or operating parameters into ICM 10A for controlling its functioning, e.g., when configured as a programmer for ICM 10A. External device 30A may be used to interrogate ICM 10A to retrieve data, including device operational data as well as physiological data accumulated in IMD memory. The interrogation may be automatic, e.g., according to a schedule, or in response to a remote or local user command. Programmers, external monitors, and consumer devices are examples of external devices 30A that may be used to interrogate ICM 10A. Examples of communication techniques used by ICM 10A and external device 30A include radiofrequency (RF) telemetry, which may be an RF link established via Bluetooth, WiFi, or medical implant communication service (MICS).

Medical device system 8A is an example of a medical device system configured to monitor the physiological response of patient 14A to physical exertion. The techniques described herein may be performed by processing circuitry of medical device system 8A, such as processing circuitry of one or both of ICM 10A and external device 30A, individually, or collectively. The techniques include detecting a plurality of exertion events of patient 14A based on a first sensed signal that varies as a function of movement of the patient 14A. The movements may include common posture transitions such as sit-stand transitions, lay-sit transitions, and walking/running events. The processing circuitry may determine a response of a physiological parameter of patient 14A to the exertion event based on second sensed signal that varies as a function of the physiological parameter. The processing circuitry may also determine that a change in the responses over time crosses a threshold, such as a number of hearts beat per minute. In some examples, ICM 10A may include or be coupled to one or more other sensors that generate one or more physiological signals, such as signals that vary based on patient motion and/or posture, blood flow, respiration, or edema.

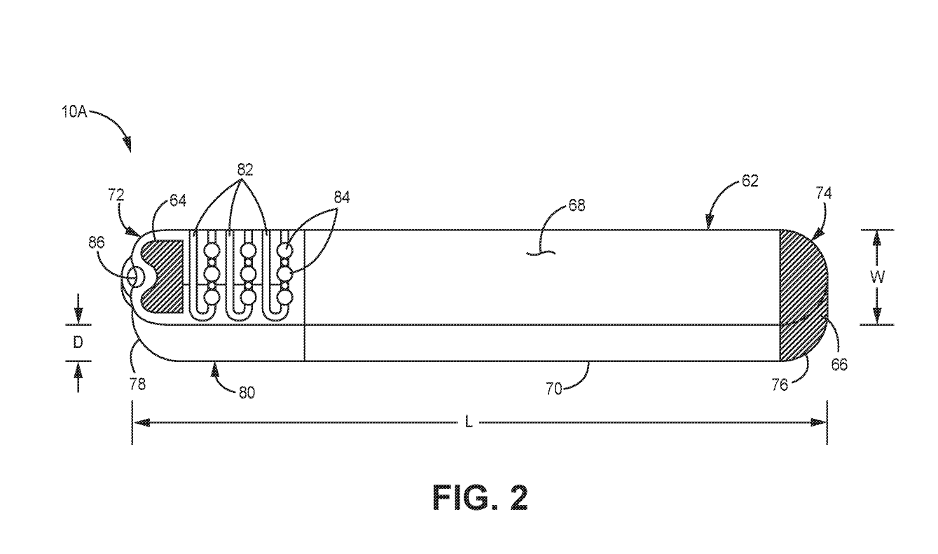

Medical device system 8A is one example of a medical device system that may be configured to implement the techniques described herein for monitoring physiological responses to exertion events. Other example medical device systems that may be configured to implement the techniques are described with respect to FIGS. 2-6. Although described herein primarily in the context of implantable medical devices monitoring signals indicating physiological parameters, a medical device system that implements the techniques described in this disclosure may additionally or alternatively include an external medical device, e.g., external cardiac monitor, and/or external pacemaker, cardioverter and/or defibrillator, configured to determine that a change in the responses to exertion events crosses a threshold and generate an alert.

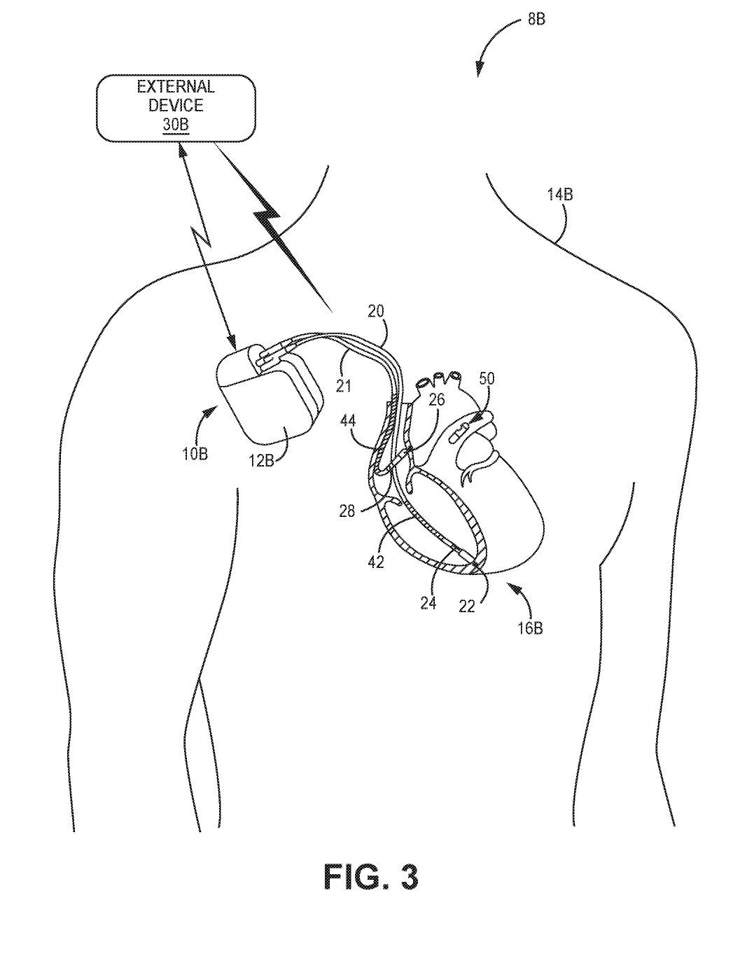

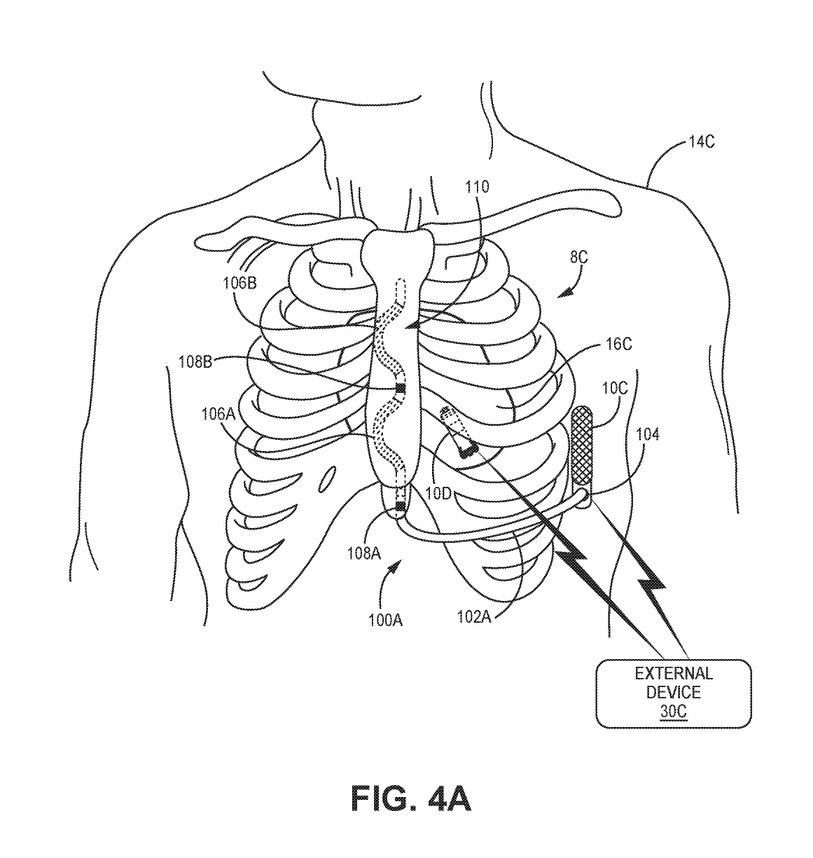

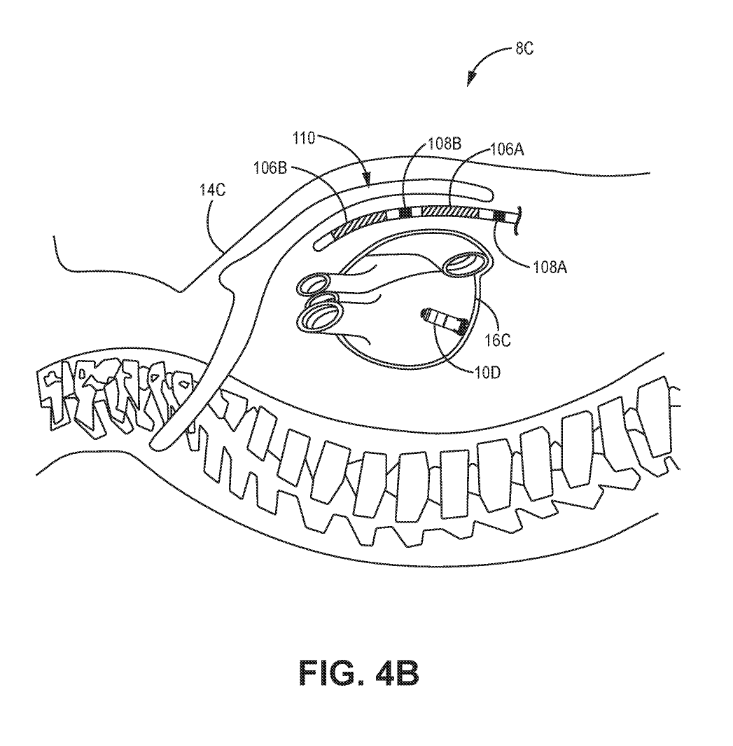

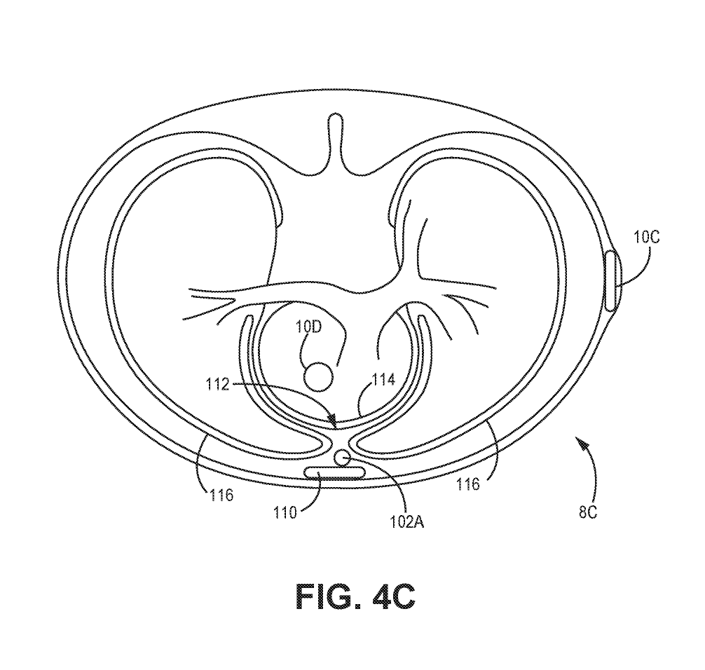

Although not illustrated in the example of FIG. 1, a medical device system configured to implement the techniques of this disclosure may include one or more implanted or external medical devices in addition to or instead of ICM 10A. For example, a medical device system may include a pressure sensing IMD, vascular ICD (e.g., ICD 10B of FIG. 3), extravascular ICD, or cardiac pacemaker (e.g., IPD 10D of FIGS. 4A-6 or a cardiac pacemaker implanted outside the heart but coupled to intracardiac or epicardial leads). One or more such devices may generate physiological signals, and include processing circuitry configured to perform, in whole or in part, the techniques described herein for monitoring physiological responses to exertion events. The implanted devices may communicate with each other and/or an external device 30, and one of the implanted or external devices may ultimately determine whether the physiological responses to exertion events crosses a threshold.

FIG. 2 is a conceptual drawing illustrating an example configuration of ICM 10A. In the example shown in FIG. 2, ICM 10A may be embodied as a monitoring device having housing 62, proximal electrode 64 and distal electrode 66. Housing 62 may further comprise first major surface 68, second major surface 70, proximal end 72, and distal end 74. Housing 62 encloses electronic circuitry located inside the ICM 10A and protects the circuitry contained therein from body fluids. Electrical feedthroughs provide electrical connection of electrodes 64 and 66.

In the example shown in FIG. 2, ICM 10A is defined by a length L, a width W and thickness or depth D and is in the form of an elongated rectangular prism wherein the length L is much larger than the width W, which in turn is larger than the depth D. In one example, the geometry of the ICM 10A—in particular a width W greater than the depth D—is selected to allow ICM 10A to be inserted under the skin of the patient using a minimally invasive procedure and to remain in the desired orientation during insertion. For example, the device shown in FIG. 2 includes radial asymmetries (notably, the rectangular shape) along the longitudinal axis that maintains the device in the proper orientation following insertion. For example, in one example the spacing between proximal electrode 64 and distal electrode 66 may range from 30 millimeters (mm) to 55 mm, 35 mm to 55 mm, and from 40 mm to 55 mm and may be any range or individual spacing from 25 mm to 60 mm. In addition, ICM 10A may have a length L that ranges from 30 mm to about 70 mm. In other examples, the length L may range from 40 mm to 60 mm, 45 mm to 60 mm and may be any length or range of lengths between about 30 mm and about 70 mm. In addition, the width W of major surface 68 may range from 3 mm to 10 mm and may be any single or range of widths between 3 mm and 10 mm. The thickness of depth D of ICM 10A may range from 2 mm to 9 mm. In other examples, the depth D of ICM 10A may range from 2 mm to 5 mm and may be any single or range of depths from 2 mm to 9 mm. In addition, ICM 10A according to an example of the present disclosure is has a geometry and size designed for ease of implant and patient comfort. Examples of ICM 10A described in this disclosure may have a volume of three cubic centimeters (cm) or less, 1.5 cubic cm or less or any volume between three and 1.5 cubic centimeters. In addition, in the example shown in FIG. 2, proximal end 72 and distal end 74 are rounded to reduce discomfort and irritation to surrounding tissue once inserted under the skin of the patient. ICM 10A, including instrument and method for inserting ICM 10A is described, for example, in U.S. Patent Publication No. 2014/0276928, incorporated herein by reference in its entirety.

In the example shown in FIG. 2, once inserted within the patient, the first major surface 68 faces outward, toward the skin of the patient while the second major surface 70 is located opposite the first major surface 68. Consequently, the first and second major surfaces may face in directions along a sagittal axis of patient 14A (FIG. 1), and this orientation may be consistently achieved upon implantation due to the dimensions of ICM 10A. Additionally, an accelerometer, or axis of an accelerometer, may be oriented along the sagittal axis.

Proximal electrode 64 and distal electrode 66 are used to sense cardiac signals, e.g. ECG signals, intra-thoracically or extra-thoracically, which may be sub-muscularly or subcutaneously. ECG signals may be stored in a memory of the ICM 10A, and ECG data may be transmitted via integrated antenna 82 to another medical device, which may be another implantable device or an external device, such as external device 30A. In some example, electrodes 64 and 66 may additionally or alternatively be used for sensing any bio-potential signal of interest, which may be, for example, an EGM, EEG, EMG, or a nerve signal, from any implanted location.

In the example shown in FIG. 2, proximal electrode 64 is in close proximity to the proximal end 72 and distal electrode 66 is in close proximity to distal end 74. In this example, distal electrode 66 is not limited to a flattened, outward facing surface, but may extend from first major surface 68 around rounded edges 76 and/or end surface 78 and onto the second major surface 70 so that the electrode 66 has a three-dimensional curved configuration. In the example shown in FIG. 2, proximal electrode 64 is located on first major surface 68 and is substantially flat, outward facing. However, in other examples proximal electrode 64 may utilize the three dimensional curved configuration of distal electrode 66, providing a three dimensional proximal electrode (not shown in this example). Similarly, in other examples distal electrode 66 may utilize a substantially flat, outward facing electrode located on first major surface 68 similar to that shown with respect to proximal electrode 64. The various electrode configurations allow for configurations in which proximal electrode 64 and distal electrode 66 are located on both first major surface 68 and second major surface 70. In other configurations, such as that shown in FIG. 2, only one of proximal electrode 64 and distal electrode 66 is located on both major surfaces 68 and 70, and in still other configurations both proximal electrode 64 and distal electrode 66 are located on one of the first major surface 68 or the second major surface 70 (i.e., proximal electrode 64 located on first major surface 68 while distal electrode 66 is located on second major surface 70). In another example, ICM 10A may include electrodes on both major surface 68 and 70 at or near the proximal and distal ends of the device, such that a total of four electrodes are included on ICM 10A. Electrodes 64 and 66 may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 2, proximal end 72 includes a header assembly 80 that includes one or more of proximal electrode 64, integrated antenna 82, anti-migration projections 84, and/or suture hole 86. Integrated antenna 82 is located on the same major surface (i.e., first major surface 68) as proximal electrode 64 and is also included as part of header assembly 80. Integrated antenna 82 allows ICM 10A to transmit and/or receive data. In other examples, integrated antenna 82 may be formed on the opposite major surface as proximal electrode 64, or may be incorporated within the housing 62 of ICM 10A. In the example shown in FIG. 2, anti-migration projections 84 are located adjacent to integrated antenna 82 and protrude away from first major surface 68 to prevent longitudinal movement of the device. In the example shown in FIG. 2, anti-migration projections 84 includes a plurality (e.g., nine) small bumps or protrusions extending away from first major surface 68. As discussed above, in other examples anti-migration projections 84 may be located on the opposite major surface as proximal electrode 64 and/or integrated antenna 82. In addition, in the example shown in FIG. 2 header assembly 80 includes suture hole 86, which provides another means of securing ICM 10A to the patient to prevent movement following insert. In the example shown, suture hole 86 is located adjacent to proximal electrode 64. In one example, header assembly 80 is a molded header assembly made from a polymeric or plastic material, which may be integrated or separable from the main portion of ICM 10A.

FIG. 3 is a conceptual drawing illustrating another example medical device system 8B in conjunction with a patient 14B. Medical device system 8B is another example of a medical device system configured to implement the techniques described herein for monitoring the health of a patient by measuring physiological responses to exertion events. In the illustrated example, medical device system 8B includes an IMD 10B and an external device 30B. IMD 10B may be an implantable cardioverter-defibrillator (ICD) capable of delivering pacing, cardioversion and defibrillation therapy to the heart 16B of a patient 14B, and will be referred to as ICD 10B hereafter. ICD 10B may monitor a physiological parameter such as heart rate, and ICD 10B may measure a change in the physiological parameter in response to an exertion event, such as a sit-stand transition.

ICD 10B may acquire cardiac electrogram (EGM) signals from patient 14B and to deliver therapy in response to the acquired data. Medical device system 8B may employ a dual chamber ICD configuration or may include one or more additional leads, such as a coronary sinus lead extending into the right atrium, through the coronary sinus and into a cardiac vein to position electrodes along the left ventricle (LV) for sensing LV EGM signals and delivering pacing pulses to the LV. In other examples, a medical device system may be a single chamber system.

ICD 10B may be coupled to a ventricular lead 20 and an atrial lead 21. Ventricular lead 20 and atrial lead 21 are electrically coupled to ICD 10B and extend into the patient's heart 16B. Ventricular lead 20 includes electrodes 22 and 24 shown positioned on the lead in the patient's right ventricle (RV) for sensing ventricular EGM signals and pacing in the RV. Atrial lead 21 includes electrodes 26 and 28 positioned on the lead in the patient's right atrium (RA) for sensing atrial EGM signals and pacing in the RA.

Ventricular lead 20 additionally carries a high voltage coil electrode 42, and atrial lead 21 carries a high voltage coil electrode 44, used to deliver cardioversion and defibrillation shocks. The term “anti-tachyarrhythmia shock” may be used herein to refer to both cardioversion shocks and defibrillation shocks. In other examples, ventricular lead 20 may carry both of high voltage coil electrodes 42 and 44, or may carry a high voltage coil electrode in addition to those illustrated in the example of FIG. 3.

ICD 10B may use both ventricular lead 20 and atrial lead 21 to acquire cardiac electrogram (EGM) signals from patient 14B and to deliver therapy in response to the acquired data. Medical device system 8B is shown as having a dual chamber ICD configuration, but other examples may include one or more additional leads, such as a coronary sinus lead extending into the right atrium, through the coronary sinus and into a cardiac vein to position electrodes along the left ventricle (LV) for sensing LV EGM signals and delivering pacing pulses to the LV. In other examples, a medical device system may be a single chamber system, or otherwise not include atrial lead 21.

Processing circuitry, sensing circuitry, and other circuitry configured for performing the techniques described herein are housed within a sealed housing 12B. Housing 12B (or a portion thereof) may be conductive so as to serve as an electrode for pacing or sensing or as an active electrode during defibrillation. As such, housing 12B is also referred to herein as “housing electrode” 12B.

ICD 10B may transmit EGM signal data and cardiac rhythm episode data acquired by ICD 10B, as well as data regarding delivery of therapy by ICD 10B, to an external device 30B. External device 30B may be a computing device, e.g., used in a home, ambulatory, clinic, or hospital setting, to communicate with ICD 10B via wireless telemetry. External device 30B may be coupled to a remote patient monitoring system, such as Carelink®, available from Medtronic plc, of Dublin, Ireland. External device 30B may be, as examples, a programmer, external monitor, or consumer device, e.g., smart phone.

External device 30B may be used to program commands or operating parameters into ICD 10B for controlling its functioning, e.g., when configured as a programmer for ICD 10B. External device 30B may be used to interrogate ICD 10B to retrieve data, including device operational data as well as physiological data accumulated in IMD memory. The interrogation may be automatic, e.g., according to a schedule, or in response to a remote or local user command. Programmers, external monitors, and consumer devices are examples of external devices 30B that may be used to interrogate ICD 10B. Examples of communication techniques used by ICD 10B and external device 30B include radiofrequency (RF) telemetry, which may be an RF link established via Bluetooth, WiFi, or medical implant communication service (MICS).

In some examples, as illustrated in FIG. 3, medical device system 8B may also include a pressure-sensing IMD 50. In the illustrated example, pressure-sensing IMD 50 is implanted in the pulmonary artery of patient 14B. In some examples, one or more pressure-sensing IMDs 50 may additionally or alternatively be implanted within a chamber of heart 16B, or generally at other locations in the circulatory system.

In one example, pressure-sensing IMD 50 is configured to sense blood pressure of patient 14B. For example, pressure-sensing IMD 50 may be arranged in the pulmonary artery and be configured to sense the pressure of blood flowing from the right ventricle outflow tract (RVOT) from the right ventricle through the pulmonary valve to the pulmonary artery. Pressure-sensing IMD 50 may therefore directly measure the pulmonary artery diastolic pressure (PAD) of patient 14B. The PAD value is a pressure value that can be employed in patient monitoring. For example, PAD may be used as a basis for evaluating congestive heart failure in a patient.

In other examples, however, pressure-sensing IMD 50 may be employed to measure blood pressure values other than PAD. For example, pressure-sensing IMD 50 may be arranged in right ventricle 28 of heart 16B to sense RV systolic or diastolic pressure, or may sense systolic or diastolic pressures at other locations of the cardiovascular system, such as within the pulmonary artery. As shown in FIG. 1, pressure-sensing IMD 50 is positioned in the main trunk of pulmonary artery 39. In other examples, a sensor, such as pressure-sensing IMD 50 may be either positioned in the right or left pulmonary artery beyond the bifurcation of the pulmonary artery.

Moreover, the placement of pressure-sensing IMD 50 is not restricted necessarily to the pulmonary side of the circulation. Pressure-sensing IMD 50 could potentially be placed in the systemic side of the circulation. For example, under certain conditions and with appropriate safety measures, pressure-sensing IMD 50 could even be placed in the left atrium, left ventricle, or aorta. Additionally, pressure-sensing IMD 50 is not restricted to placement within the cardiovascular system. For example, the pressure-sensing IMD 50 might be placed in the renal circulation. Placement of pressure-sensing IMD 50 in the renal circulation may be beneficial, for example, to monitor the degree of renal insufficiency in the patient based on the monitoring of pressure or some other indication of renal circulation by pressure-sensing IMD 50.

In some examples, pressure-sensing IMD 50 includes a pressure sensor configured to respond to the absolute pressure inside the pulmonary artery of patient 14B. Pressure-sensing IMD 50 may be, in such examples, any of a number of different types of pressure sensors. One form of pressure sensor that may be useful for measuring blood pressure is a capacitive pressure sensor. Another example pressure sensor is an inductive sensor. In some examples, pressure-sensing IMD 50 may also comprise a piezoelectric or piezoresistive pressure transducer. In some examples, pressure-sensing IMD 50 may comprise a flow sensor.

In one example, pressure-sensing IMD 50 comprises a leadless pressure sensor including capacitive pressure sensing elements configured to measure blood pressure within the pulmonary artery. Pressure-sensing IMD 50 may be in wireless communication with ICD 10B and/or external device 30B, e.g., in order to transmit blood pressure measurements to one or both of the devices. Pressure-sensing IMD 50 may employ, e.g., radio frequency (RF) or other telemetry techniques for communicating with ICD 10B and other devices, including, e.g., external device 30B. In another example, pressure-sensing IMD 50 may include a tissue conductance communication (TCC) system by which the device employs tissue of patient 14B as an electrical communication medium over which to send and receive information to and from ICD 10B and/or external device 30B.

External device 30B may be configured in a manner substantially similar to that described above with respect to external device 30A and FIG. 1. External device 30B may wirelessly communicate with ICD 10B, e.g., to program the functionality of the ICD, and to retrieve recorded physiological signals and/or patient parameter values or other data derived from such signals from the ICD. Both ICD 10B and external device 30B include processing circuitry, and the processing circuitry of either or both device may perform the techniques described herein, such as detecting a plurality of exertion events of patient 14B based a first sensed signal that varies as a function of movement of the patient 14B, determining a response of a physiological parameter of patient 14B to the exertion event based on second sensed signal that varies as a function of the physiological parameter, and determining whether a change in the responses over time crosses a threshold.

Medical device system 8B is an example of a medical device system configured to monitor the physiological response to exertion events. The techniques may be performed by processing circuitry of medical device system 8B, such as processing circuitry of one or both of ICD 10B and external device 30B, individually, or collectively.

The techniques include determining a respective value for each of a plurality of parameters of a patient, e.g., physiological and/or pathophysiological, during each of a plurality of periods, which may be at least one hour, such as between approximately one day and approximately three days, e.g., in one example, approximately one day. The processing circuitry may determine the values of at least some the patient parameters based on physiological signals generated by sensing circuitry of one or both of ICD 10B and pressure-sensing IMD 50, such as a cardiac EGM signal generated by sensing circuitry of ICD 10B, or a pulmonary artery or other cardiovascular pressure signal generated by pressure-sensing IMD 50. In some examples, one or both of ICD 10B and pressure-sensing IMD 50 may include or be coupled to one or more other sensors that generate one or more physiological signals, such as signals that vary based on patient motion and/or posture, blood flow, respiration, or edema. The processing circuitry may determine other patient parameters based on therapy delivered by ICD 10B, such as patient parameters indicating the extent to which patient 14B is dependent on pacing, e.g., a percentage of time or other characterization of amount of pacing delivered to the patient.

In some examples, the processing circuitry of medical device system 8B detects a plurality of exertion events for patient 14B based on a first sensed signal, such as one or more accelerometer signals. The exertion events may comprise common posture transitions such as sit-stand transitions, lay-sit transitions, stand-walk transitions, walking events, or any physical exertion event that may have an effect on a physiological parameter. The exertion events may also comprise periods of activity that exceed a threshold duration, such as walking for more than twenty seconds or more than twenty steps. The processing circuitry may determine the response of a physiological parameter based on a second sensed signal. The physiological parameter may comprise heart rate, blood pressure, respiration, or any other measurable physiological parameter. The processing circuitry may determine that a change in the responses over time crosses a threshold. For example, the processing circuitry may determine that, in response to a sit-stand transition, the heart rate of patient 14B increased by a number or percentage of beats per minute. The processing circuitry may determine that this response crosses, e.g., exceeds and/or falls below, a threshold or threshold range for responses, which may be determined based on one or more prior responses, e.g., a median and baseline variability of prior responses. Based on determining that the change crosses the threshold, the processing circuitry may generate an alert to a user through a communication module or a user interface.

Medical device system 8B is one example of a medical device system that may be configured to implement the techniques described herein for monitoring the physiological response to exertion events. Other example medical device systems that may be configured to implement the techniques are described with respect to FIGS. 4A-6. Although described herein primarily in the context of implantable medical devices generating physiological signals and, in some examples, delivering therapy, a medical device system that implements the techniques described in this disclosure may additionally or alternatively include an external medical device, e.g., external cardiac monitor, and/or external pacemaker, cardioverter and/or defibrillator, configured to generate one or more of the physiological signals described herein, monitor physiological responses and/or generate an alert.

FIGS. 4A-4C are front-view, side-view, and top-view conceptual drawings, respectively, illustrating another example medical device system 8C in conjunction with a patient 14C. Medical device system 8C is another example of a medical device system configured to implement the techniques described herein for monitoring physiological responses to exertion events, and responsively generating an alert indicating that a physiological response crosses a threshold.

In the illustrated example, medical device system 8C includes an extracardiovascular ICD system 100A implanted within a patient 14C. ICD system 100A includes an IMD 10C, which is an ICD and is referred to hereafter as ICD 10C, connected to at least one implantable cardiac defibrillation lead 102A. ICD 10C may be configured to deliver high-energy cardioversion or defibrillation pulses to a patient's heart 16C when atrial or ventricular fibrillation is detected. Cardioversion shocks are typically delivered in synchrony with a detected R-wave when fibrillation detection criteria are met. Defibrillation shocks are typically delivered when fibrillation criteria are met, and the R-wave cannot be discerned from signals sensed by ICD 10C.

ICD 10C is implanted subcutaneously or submuscularly on the left side of patient 14C above the ribcage. Defibrillation lead 102A may be implanted at least partially in a substernal location, e.g., between the ribcage and/or sternum 110 and heart 16C. In one such configuration, a proximal portion of lead 102A extends subcutaneously from ICD 10C toward sternum 110 and a distal portion of lead 102A extends superior under or below the sternum 110 in the anterior mediastinum 112 (FIG. 4C). The anterior mediastinum 112 is bounded laterally by the pleurae 116, posteriorly by the pericardium 114 (FIG. 4C), and anteriorly by the sternum 110. In some instances, the anterior wall of the anterior mediastinum may also be formed by the transversus thoracis and one or more costal cartilages. The anterior mediastinum includes a quantity of loose connective tissue (such as areolar tissue), some lymph vessels, lymph glands, substernal musculature (e.g., transverse thoracic muscle), branches of the internal thoracic artery, and the internal thoracic vein. In one example, the distal portion of lead 102A extends along the posterior side of the sternum 110 substantially within the loose connective tissue and/or substernal musculature of the anterior mediastinum. Lead 102A may be at least partially implanted in other intrathoracic locations, e.g., other non-vascular, extra-pericardial locations, including the gap, tissue, or other anatomical features around the perimeter of and adjacent to, but not attached to, the pericardium or other portion of the heart and not above the sternum 110 or ribcage.

In other examples, lead 102A may be implanted at other extracardiovascular locations. For example, defibrillation lead 102A may extend subcutaneously above the ribcage from ICD 10C toward a center of the torso of patient 14C, bend or turn near the center of the torso, and extend subcutaneously superior above the ribcage and/or sternum 110. Defibrillation lead 102A may be offset laterally to the left or the right of the sternum 110 or located over the sternum 110. Defibrillation lead 102A may extend substantially parallel to the sternum 110 or be angled lateral from the sternum 110 at either the proximal or distal end.

Defibrillation lead 102A includes an insulative lead body having a proximal end that includes a connector 104 configured to be connected to ICD 10C and a distal portion that includes one or more electrodes. Defibrillation lead 102A also includes one or more conductors that form an electrically conductive path within the lead body and interconnect the electrical connector and respective ones of the electrodes.

Defibrillation lead 102A includes a defibrillation electrode that includes two sections or segments 106A and 106B, collectively (or alternatively) defibrillation electrode 106. The defibrillation electrode 106 is toward the distal portion of defibrillation lead 102A, e.g., toward the portion of defibrillation lead 102A extending along the sternum 110. Defibrillation lead 102A is placed below and/or along sternum 110 such that a therapy vector between defibrillation electrodes 106A or 106B and a housing electrode formed by or on ICD 10C (or other second electrode of the therapy vector) is substantially across a ventricle of heart 16C. The therapy vector may, in one example, be viewed as a line that extends from a point on defibrillation electrode 106 (e.g., a center of one of the defibrillation electrode sections 106A or 106B) to a point on the housing electrode of ICD 10C. Defibrillation electrode 106 may, in one example, be an elongated coil electrode.

Defibrillation lead 102A may also include one or more sensing electrodes, such as sensing electrodes 108A and 108B (individually or collectively, “sensing electrode(s) 108”), located along the distal portion of defibrillation lead 102A. In the example illustrated in FIG. 4A and FIG. 4B, sensing electrodes 108A and 108B are separated from one another by defibrillation electrode 106A. In other examples, however, sensing electrodes 108A and 108B may be both distal of defibrillation electrode 106 or both proximal of defibrillation electrode 106. In other examples, lead 102A may include more or fewer electrodes at various locations proximal and/or distal to defibrillation electrode 106. In the same or different examples, ICD 10C may include one or more electrodes on another lead (not shown).

ICD system 100A may sense electrical signals via one or more sensing vectors that include combinations of electrodes 108A and 108B and the housing electrode of ICD 10C. In some instances, ICD 10C may sense cardiac electrical signals using a sensing vector that includes one of the defibrillation electrode sections 106A and 106B and one of sensing electrodes 108A and 108B or the housing electrode of ICD 9. The sensed electrical intrinsic signals may include electrical signals generated by cardiac muscle and indicative of depolarizations and repolarizations of heart 16C at various times during the cardiac cycle. ICD 10C analyzes the electrical signals sensed by the one or more sensing vectors to detect a physiological parameter, such as heart rate, blood pressure, respiration, and the like. ICD 10C, e.g., using one or more accelerometers within ICD 10C, may detect a plurality of exertion events based on first sensed signals from the accelerometers. The first sensed signals may vary as a function of the movement of patient 14C. The processing circuitry within ICD 10C may determine a physiological response to each exertion event and determine whether a change in the responses over time crosses a threshold. The change over time may be a trend in the measurements or a single measurement that crosses a threshold, which may be determined based on past measurements of the physiological response. For example, the heart-rate responses to sit-stand transitions may increase gradually over time for patient 14C. If the trend in heart-rate responses, such as a trend in the mean or median heart-rate response to a sit-stand transition, exceeds and/or falls below a threshold or threshold range, which may be determined based on baseline or other past responses, ICD 10C may generate an alert.

Medical device system 8C also includes an IMD 10D, which is implanted within heart 16C and configured to deliver cardiac pacing to the heart, e.g., is an intracardiac pacing device (IPD). IMD 10D is referred to as IPD 10D hereafter. In the illustrated example, IPD 10D is implanted within the right ventricle of heart 16C. However, in other examples, system 8C may additionally or alternatively include one or more IPDs 10D within other chambers of heart 16C, or similarly configured pacing devices attached to an external surface of heart 16C (e.g., in contact with the epicardium) such that the pacing device is disposed outside of heart 16C.

IPD 10D may be configured to sense electrical activity of heart 16C and deliver pacing therapy, e.g., bradycardia pacing therapy, cardiac resynchronization therapy (CRT), anti-tachycardia pacing (ATP) therapy, and/or post-shock pacing, to heart 16C. IPD 10D may be attached to an interior wall of heart 16C via one or more fixation elements that penetrate the tissue. These fixation elements may secure IPD 10D to the cardiac tissue and retain an electrode (e.g., a cathode or an anode) in contact with the cardiac tissue.

IPD 10D may be capable sensing electrical signals using the electrodes carried on the housing of IPD 10D. These electrical signals may be electrical signals generated by cardiac muscle and indicative of depolarizations and repolarizations of heart 16C at various times during the cardiac cycle. IPD 10D may analyze the sensed electrical signals to detect bradycardia and tachyarrhythmias, such as ventricular tachycardia or ventricular fibrillation. In response to detecting bradycardia, IPD 10D may deliver bradycardia pacing via the electrodes of IPD 10D. In response to detecting tachyarrhythmia, IPD 10D may, e.g., depending on the type of tachyarrhythmia, deliver ATP therapy via the electrodes of IPD 10D. In some examples, IPD 10D may deliver post-shock pacing in response to determining that another medical device, e.g., ICD 10C, delivered an anti-tachyarrhythmia shock.

IPD 10D and ICD 10C may be configured to coordinate their arrhythmia detection and treatment activities. In some examples IPD 10D and ICD 10C may be configured to operate completely independently of one another. In such a case, IPD 10D and ICD 10C are not capable of establishing telemetry communication sessions with one another to exchange information about sensing and/or therapy using one-way or two-way communication. Instead, each of IPD 10D and ICD 10C analyze the data sensed via their respective electrodes to make tachyarrhythmia detection and/or therapy decisions. As such, each device does not know if the other will detect the tachyarrhythmia, if or when it will provide therapy, and the like. In some examples, IPD 10D may be configured to detect anti-tachyarrhythmia shocks delivered by ICD system 100A, which may improve the coordination of therapy between subcutaneous ICD 10C and IPD 10D without requiring device-to-device communication. In this manner, IPD 10D may coordinate the delivery of cardiac stimulation therapy, including the termination of ATP and the initiation of the delivery of post-shock pacing, with the application of an anti-tachyarrhythmia shock merely through the detection of defibrillation pulses and without the need to communicate with the defibrillation device applying the anti-tachyarrhythmia shock.

In other examples, IPD 10D and ICD 10C may engage in communication to facilitate the appropriate detection of arrhythmias and/or delivery of therapy. The communication may include one-way communication in which one device is configured to transmit communication messages and the other device is configured to receive those messages. The communication may instead include two-way communication in which each device is configured to transmit and receive communication messages. Two-way communication and coordination of the delivery of patient therapies between IPD 10D and ICD 10C is described in commonly-assigned U.S. patent application Ser. No. 13/756,085, titled, “SYSTEMS AND METHODS FOR LEADLESS PACING AND SHOCK THERAPY,” filed Jan. 31, 2013, the entire content of which is incorporated by reference herein.

External device 30C may be configured substantially similarly to external device 30A described above with respect to FIG. 1. External device 30C may be configured to communicate with one or both of ICD 10C and IPD 10D. In examples where external device 30C only communicates with one of ICD 10C and IPD 10D, the non-communicative device may receive instructions from or transmit data to the device in communication with external device 30C. In some examples, a user may interact with device 30C remotely via a networked computing device. The user may interact with external device 30C to communicate with IPD 10D and/or ICD 10C.

For example, the user may interact with external device 30C to send an interrogation request and retrieve sensed physiological data or therapy delivery data stored by one or both of ICD 10C and IPD 10D, and program or update therapy parameters that define therapy, or perform any other activities with respect to ICD 10C and IPD 10D. Although the user is a physician, technician, surgeon, electrophysiologist, or other healthcare professional, the user may be patient 14C in some examples. For example, external device 21 may allow a user to program any coefficients, weighting factors, or techniques for determining difference metrics, scores, and/or thresholds, or other data described herein as being used by a medical device system to determine whether a physiological response crosses a threshold.

Although FIGS. 4A-4C are shown or described in the context of IPD 10D and extracardiovascular ICD system 100A that includes lead 102A with a substernally placed distal portion, techniques in accordance with one or more aspects of the present disclosure may be applicable to other coexistent systems. For example, an extracardiovascular ICD system may include a lead having a distal portion that is implanted subcutaneously above the sternum (or other location) instead of being implanted substernally. As another example, instead of an IPD, a pacing system may be implanted having a pacemaker and one or more leads connected to and extending from the pacemaker into one or more chambers of the heart or attached to the outside of the heart to provide pacing therapy to the one or more chambers. As such, the example of FIGS. 4A-4C is illustrated for example purposes only and should not be considered limiting of the techniques described herein.

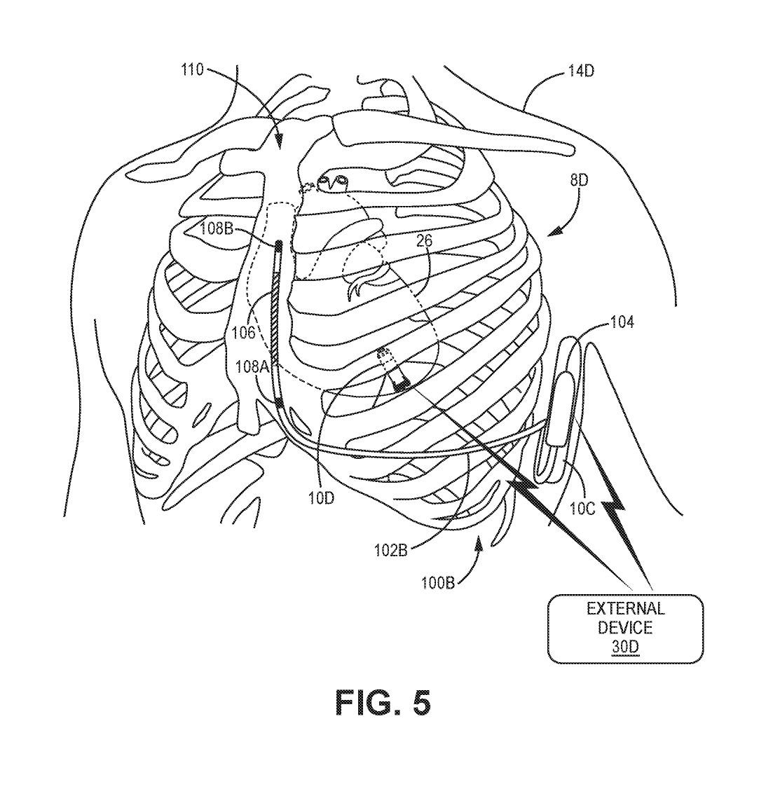

FIG. 5 is a conceptual drawing illustrating another example medical device system 8D that includes an extracardiovascular ICD system 100B and IPD 10D implanted within a patient. Medical device system 8B may be configured to perform any of the techniques described herein with respect to medical device system 8C of FIGS. 4A-4C. Components with like numbers in FIGS. 4A-4C and FIG. 5 may be similarly configured and provide similar functionality.

In the example of FIG. 5, extracardiovascular ICD system 100B includes ICD 10C coupled to a defibrillation lead 102B. Unlike defibrillation lead 102A of FIGS. 4A-4C, defibrillation lead 102B extends subcutaneously above the ribcage from ICD 10C. In the illustrated example, defibrillation lead 102B extends toward a center of the torso of patient 14D, bends or turns near the center of the torso, and extends subcutaneously superior above the ribcage and/or sternum 110. Defibrillation lead 102B may be offset laterally to the left or the right of sternum 110 or located over sternum 110. Defibrillation lead 102B may extend substantially parallel to sternum 102 or be angled lateral from the sternum at either the proximal or distal end.

Defibrillation lead 102B includes an insulative lead body having a proximal end that includes a connector 104 configured to be connected to ICD 10C and a distal portion that includes one or more electrodes. Defibrillation lead 102B also includes one or more conductors that form an electrically conductive path within the lead body and interconnect the electrical connector and respective ones of the electrodes. In the illustrated example, defibrillation lead 102B includes a single defibrillation electrode 106 toward the distal portion of defibrillation lead 102B, e.g., toward the portion of defibrillation lead 102B extending along sternum 110. Defibrillation lead 102B is placed along sternum 110 such that a therapy vector between defibrillation electrode 106 and a housing electrode formed by or on ICD 10C (or other second electrode of the therapy vector) is substantially across a ventricle of heart 16D.

Defibrillation lead 102B may also include one or more sensing electrodes, such as sensing electrodes 108A and 108B, located along the distal portion of defibrillation lead 102B. In the example illustrated in FIG. 5, sensing electrodes 108A and 108B are separated from one another by defibrillation electrode 106. In other examples, however, sensing electrodes 108A and 108B may be both distal of defibrillation electrode 106 or both proximal of defibrillation electrode 106. In other examples, lead 102B may include more or fewer electrodes at various locations proximal and/or distal to defibrillation electrode 106, and lead 102B may include multiple defibrillation electrodes, e.g., defibrillation electrodes 106A and 106B as illustrated in the example of FIGS. 4A-4C.

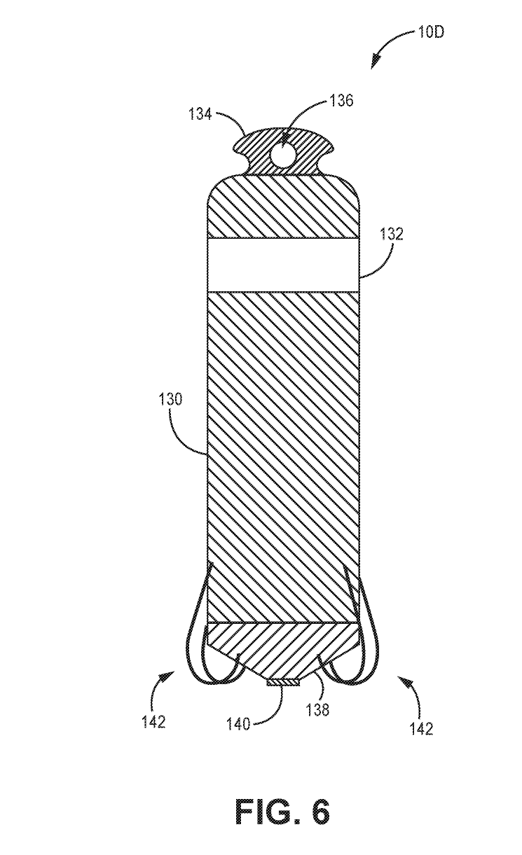

FIG. 6 is a conceptual drawing illustrating an example configuration of IPD 10D. As shown in FIG. 6, IPD 10D includes case 130, cap 138, electrode 140, electrode 132, fixation mechanisms 142, flange 134, and opening 136. Together, case 130 and cap 138 may be considered the housing of IPD 10D. In this manner, case 130 and cap 138 may enclose and protect the various electrical components, e.g., circuitry, within IPD 10D. Case 130 may enclose substantially all of the electrical components, and cap 138 may seal case 130 and create the hermetically sealed housing of IPD 10D. Although IPD 10D is generally described as including one or more electrodes, IPD 10D may typically include at least two electrodes (e.g., electrodes 132 and 140) to deliver an electrical signal (e.g., therapy such as cardiac pacing) and/or provide at least one sensing vector.

Electrodes 132 and 140 are carried on the housing created by case 130 and cap 138. In this manner, electrodes 132 and 140 may be considered leadless electrodes. In the example of FIG. 6, electrode 140 is disposed on the exterior surface of cap 138. Electrode 140 may be a circular electrode positioned to contact cardiac tissue upon implantation. Electrode 132 may be a ring or cylindrical electrode disposed on the exterior surface of case 130. Both case 130 and cap 138 may be electrically insulating.

Electrode 140 may be used as a cathode and electrode 132 may be used as an anode, or vice versa, for delivering cardiac pacing such as bradycardia pacing, CRT, ATP, or post-shock pacing. However, electrodes 132 and 140 may be used in any stimulation configuration. In addition, electrodes 132 and 140 may be used to detect intrinsic electrical signals from cardiac muscle.

Fixation mechanisms 142 may attach IPD 10D to cardiac tissue. Fixation mechanisms 142 may be active fixation tines, screws, clamps, adhesive members, or any other mechanisms for attaching a device to tissue. As shown in the example of FIG. 6, fixation mechanisms 142 may be constructed of a memory material, such as a shape memory alloy (e.g., nickel titanium), that retains a preformed shape. During implantation, fixation mechanisms 142 may be flexed forward to pierce tissue and allowed to flex back towards case 130. In this manner, fixation mechanisms 142 may be embedded within the target tissue.

Flange 144 may be provided on one end of case 130 to enable tethering or extraction of IPD 10D. For example, a suture or other device may be inserted around flange 144 and/or through opening 146 and attached to tissue. In this manner, flange 144 may provide a secondary attachment structure to tether or retain IPD 10D within heart 16C (or 16D) if fixation mechanisms 142 fail. Flange 144 and/or opening 146 may also be used to extract IPD 10D once the IPD needs to be explanted (or removed) from patient 14D if such action is deemed necessary.

IPD 10D is one example of a pacing device configured to implement the techniques of this disclosure. However, other implantable medical devices may be used to perform the same or similar functions as IPD 10D. For example, an IPD may include a small housing that carries an electrode, similar to IPD 10D, and be configured to be implanted within a chamber of a heart 16. The IPD may also include one or more relatively short leads configured to place one or more respective additional electrodes at another location within the same chamber of the heart or a different chamber of the heart. In this manner, the housing of the IPD may not carry all of the electrodes used to perform functions described herein with respect to IPD 10D. In other examples, each electrode of the IPD may be carried by one or more leads (e.g., the housing of the IPD may not carry any of the electrodes). In some examples, an IPD or other pacing device may include or be coupled to three or more electrodes, where each electrode may deliver therapy and/or detect intrinsic signals.

In another example, a pacing device may be configured to be implanted external to the heart, e.g., near or attached to the epicardium of the heart. An electrode carried by the housing of the pacing may be placed in contact with the epicardium and/or one or more electrodes of leads coupled to the pacing may be placed in contact with the epicardium at locations sufficient to provide cardiac pacing. In still other examples, a pacing device configured to perform the techniques described herein may be implanted subcutaneously or submuscularly, and connected to one or more intracardiac leads carrying one or more electrodes.

Referring back to FIGS. 4A-5, medical device systems 8C and 8D are examples of medical device systems configured to determine whether a physiological response crosses a threshold. The techniques may be performed by processing circuitry of medical device system 8C or 8D, such as processing circuitry of one or more of ICD 10C, IPD 10D, and external device 30C or 30D, individually, or collectively. Although the example medical devices systems 8C and 8D of FIGS. 4A-5 are illustrated as including both ICD 10C and IPD 10D, other examples may include only one of ICD 10C or IPD 10D, alone, or in combination with other implanted or external devices.

The techniques include determining a respective value for each of a plurality of patient parameters of a patent during each of a plurality of periods, which may be at least one hour, such as approximately one day. The processing circuitry may determine the values of at least some the patient parameters based on physiological signals generated by sensing circuitry of one or both of ICD 10C and IPD 10D, such as cardiac EGM signals generated by sensing circuitry of the IMDs. In some examples, one or both of ICD 10C and IPD 10D may include or be coupled to one or more other sensors that generate one or more physiological signals, such as signals that vary based on patient motion and/or posture, blood flow, blood pressure (e.g., systems 8C and 8D may include pressure sensing IMD 50, described above with respect to FIG. 1), respiration, or edema. The processing circuitry may determine other patient parameters based on therapies delivered by ICD 10C and/or IPD 10D, such as patient parameters indicating the extent to which patient 14C or 14D is dependent on pacing, e.g., a percentage of time or other characterization of amount of pacing delivered to the patient, or the number of anti-tachyarrhythmia therapies delivered to the patient.

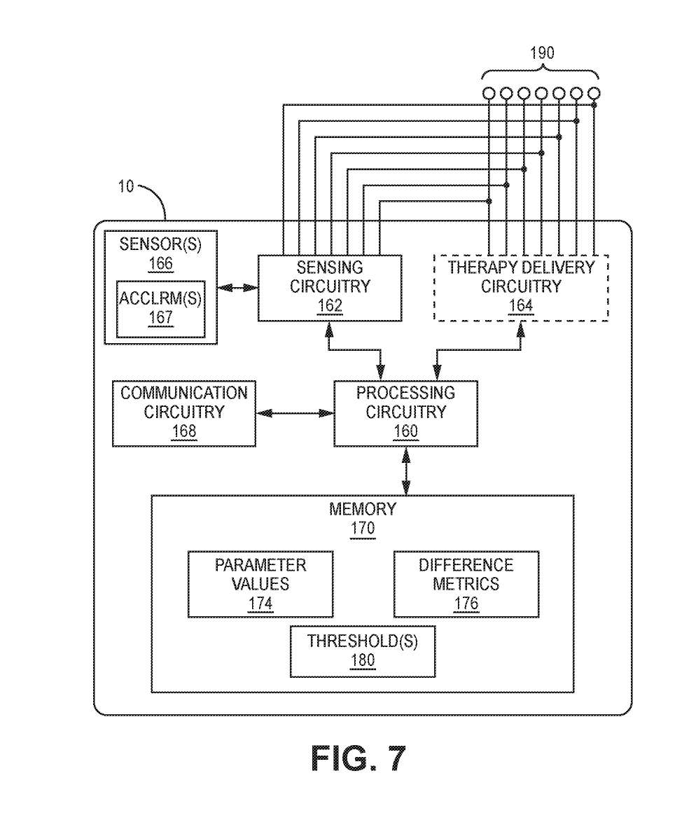

FIG. 7 is a functional block diagram illustrating an example configuration of an IMD 10. IMD 10 may correspond to any of ICM 10A, ICD 10B, ICD 10C, IPD 10D, or another IMD configured to implement the techniques for determining whether a physiological response crosses a threshold as described in this disclosure. In the illustrated example, IMD 10 includes processing circuitry 160 and an associated memory 170, sensing circuitry 162, therapy delivery circuitry 164, one or more sensors 166, and communication circuitry 168. However, ICD 10A, ICM 10B, ICD 10C, and IPD 10D need not include all of these components, or may include additional components. For example, ICM 10A may not include therapy delivery circuitry 164, in some examples.

Memory 170 includes computer-readable instructions that, when executed by processing circuitry 160, cause IMD 10 and processing circuitry 160 to perform various functions attributed to IMD 10 and processing circuitry 160 herein (e.g., determining patient parameter values, difference metrics, scores and thresholds, and determining whether to generate an alert indicating that a physiological response crosses a threshold). Memory 170 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media. Memory 170 may store threshold(s) for physiological parameters such as maximum changes and minimum changes. Memory 170 may also store data indicating changes in physiological parameters over time in response to exertion events.

Processing circuitry 160 may include fixed function circuitry and/or programmable processing circuitry. Processing circuitry 160 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 160 may include multiple components, such as any combination of one or more microprocessors, one or more controllers, one or more DSPs, one or more ASICs, or one or more FPGAs, as well as other discrete or integrated logic circuitry. The functions attributed to processing circuitry 160 herein may be embodied as software, firmware, hardware or any combination thereof.

Sensing circuitry 162 and therapy delivery circuitry 164 are coupled to electrodes 190. Electrodes 190 illustrated in FIG. 7 may correspond to, for example: electrodes 12, 22, 24, 26, 28, 44, and 44 of ICD 10A (FIG. 1); electrodes 64 and 66 of ICM 10B (FIG. 3); electrodes 106, 108, and one or more housing electrodes of ICD 10C (FIGS. 4A-5); or electrodes 132 and 140 of IPD 10D (FIG. 6).

Sensing circuitry 162 monitors signals from a selected two or more of electrodes 190 in order to monitor electrical activity of heart, impedance, or other electrical phenomenon. Sensing of a cardiac electrical signal may be done to determine heart rates or heart rate variability, or to detect arrhythmias (e.g., tachyarrhythmias or bradycardia) or other electrical signals. In some examples, sensing circuitry 162 may include one or more filters and amplifiers for filtering and amplifying a signal received from electrodes 190. In some examples, sensing circuitry 162 may sense or detect physiological parameters, such as heart rate, blood pressure, respiration, and the like.

The resulting cardiac electrical signal may be passed to cardiac event detection circuitry that detects a cardiac event when the cardiac electrical signal crosses a sensing threshold. The cardiac event detection circuitry may include a rectifier, filter and/or amplifier, a sense amplifier, comparator, and/or analog-to-digital converter. Sensing circuitry 162 outputs an indication to processing circuitry 160 in response to sensing of a cardiac event (e.g., detected P-waves or R-waves).

In this manner, processing circuitry 160 may receive detected cardiac event signals corresponding to the occurrence of detected R-waves and P-waves in the respective chambers of heart. Indications of detected R-waves and P-waves may be used for detecting ventricular and/or atrial tachyarrhythmia episodes, e.g., ventricular or atrial fibrillation episodes. Some detection channels may be configured to detect cardiac events, such as P- or R-waves, and provide indications of the occurrences of such events to processing circuitry 160, e.g., as described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety.

Sensing circuitry 162 may also include a switch module to select which of the available electrodes 190 (or electrode polarities) are used to sense the heart activity. In examples with several electrodes 190, processing circuitry 160 may select the electrodes that function as sense electrodes, i.e., select the sensing configuration, via the switch module within sensing circuitry 162. Sensing circuitry 162 may also pass one or more digitized EGM signals to processing circuitry 160 for analysis, e.g., for use in cardiac rhythm discrimination.

Processing circuitry 160 may implement programmable counters. If IMD 10 is configured to generate and deliver pacing pulses to heart, such counters may control the basic time intervals associated with bradycardia pacing (e.g., DDD, VVI, DVI, VDD, AAI, DDI, DDDR, VVIR, DVIR, VDDR, AAIR, DDIR pacing) and other modes of pacing. Intervals defined by processing circuitry 160 may include atrial and ventricular pacing escape intervals, refractory periods during which sensed P-waves and R-waves are ineffective to restart timing of the escape intervals, and the pulse widths of the pacing pulses. The durations of these intervals may be determined by processing circuitry 160 in response to pacing mode parameters stored in memory 170.

Interval counters implemented by processing circuitry 160 may be reset upon sensing of R-waves and P-waves with detection channels of sensing circuitry 162, or upon the generation of pacing pulses by therapy delivery circuitry 164, and thereby control the basic timing of cardiac pacing functions, including bradycardia pacing, CRT, ATP, or post-shock pacing. The value of the count present in the interval counters when reset by sensed R-waves and P-waves may be used by processing circuitry 160 to measure the durations of R-R intervals, P-P intervals, P-R intervals and R-P intervals, which are measurements that may be stored in memory 170. Processing circuitry 160 may use the count in the interval counters to detect a tachyarrhythmia event, such as atrial fibrillation (AF), atrial tachycardia (AT), VF, or VT. These intervals may also be used to detect the overall heart rate, ventricular contraction rate, and heart rate variability. A portion of memory 170 may be configured as a plurality of recirculating buffers, capable of holding series of measured intervals, which may be analyzed by processing circuitry 160 in response to the occurrence of a pace or sense interrupt to determine whether the patient's heart is presently exhibiting atrial or ventricular tachyarrhythmia.

In some examples, an arrhythmia detection method may include any suitable tachyarrhythmia detection algorithms. In one example, processing circuitry 160 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. U.S. Pat. No. 5,755,736 to Gillberg et al. is incorporated herein by reference in their entireties. However, other arrhythmia detection methodologies, such as those methodologies that utilize timing and morphology of the electrocardiogram, may also be employed by processing circuitry 160 in other examples.

In some examples, processing circuitry 160 may determine that tachyarrhythmia has occurred by identification of shortened R-R (or P-P) interval lengths. Generally, processing circuitry 160 detects tachycardia when the interval length falls below 220 milliseconds and fibrillation when the interval length falls below 180 milliseconds. In other examples, processing circuitry 160 may detect ventricular tachycardia when the interval length falls between 330 milliseconds and ventricular fibrillation when the interval length falls below 240 milliseconds. These interval lengths are merely examples, and a user may define the interval lengths as desired, which may then be stored within memory 170. This interval length may need to be detected for a certain number of consecutive cycles, for a certain percentage of cycles within a running window, or a running average for a certain number of cardiac cycles, as examples. In other examples, additional patient parameters may be used to detect an arrhythmia. For example, processing circuitry 160 may analyze one or more morphology measurements, impedances, or any other physiological measurements to determine that patient is experiencing a tachyarrhythmia.

In addition to detecting and identifying specific types of cardiac events, e.g., cardiac depolarizations, sensing circuitry 162 may also sample the detected intrinsic signals to generate an electrogram or other time-based indication of cardiac events. Sensing circuitry 162 may include an analog-to-digital converter or other circuitry configured to sample and digitize the electrical signal sensed via electrodes 190. Processing circuitry 160 may analyze the digitized signal for a variety of purposes, including morphological identification or confirmation of tachyarrhythmia of heart. As another example, processing circuitry 160 may analyze the digitized cardiac electrogram signal to identify and measure a variety of morphological features of the signal. The morphological features of the cardiac electrogram may, in some examples, be patient parameters, and their measurements patient parameter values, used to determine whether a physiological response crosses a threshold.

In some examples, sensing circuitry 162 is configured to sense physiological signals of patient. For example, sensing circuitry 162 may be configured to sense signals that vary with changing thoracic impedance of patient 14. The thoracic impedance may vary based on fluid volume or edema in patient 14.

Sensing circuitry 162 may use any two or more of electrodes 190 to sense thoracic impedance. As the tissues within the thoracic cavity of patient 14 change in fluid content, the impedance between two electrodes may also change. For example, the impedance between a defibrillation coil electrode (42, 44, 106) and the housing electrode may be used to monitor changing thoracic impedance.

In some examples, processing circuitry 160 measured thoracic impedance values to determine a fluid index. As more fluid is retained within patient 14, e.g., edema increases, and the thoracic impedance decreases or remains relatively high, the fluid index increases. Conversely, as the thoracic impedance increases or remains relatively low, the fluid index decreases. An example system for measuring thoracic impedance and determining a fluid index is described in U.S. Patent Publication No. 2010/0030292 to Sarkar et al., entitled, “DETECTING WORSENING HEART FAILURE BASED ON IMPEDANCE MEASUREMENTS,” which published on Feb. 4, 2010 and is incorporated herein by reference in its entirety.

The thoracic impedance may also vary with patient respiration. In some examples, processing circuitry 160 may determine values of one or more respiration-related patient parameters based on thoracic impedance sensed by sensing circuitry 162. Respiration-related patient parameters may include, as examples, respiration rate, respiration depth, or the occurrence or magnitude of dyspnea or apneas.

The magnitude of the cardiac electrogram may also vary based on patient respiration, e.g., generally at a lower frequency than the cardiac cycle. In some examples, processing circuitry 160 and/or sensing circuitry 162 may filter the cardiac electrogram to emphasize the respiration component of the signal. Processing circuitry 160 may analyze the filtered cardiac electrogram signal to determine values of respiration-related patient parameters.

In the example of FIG. 7, IMD 10 includes one or more sensors 166 coupled to sensing circuitry 162. Although illustrated in FIG. 7 as included within IMD 10, one or more of sensors 166 may be external to IMD 10, e.g., coupled to IMD 10 via one or more leads, or configured to wirelessly communicate with IMD 10. In some examples, sensors 166 transduce a signal indicative of a patient parameter, which may be amplified, filtered, or otherwise processed by sensing circuitry 162. In such examples, processing circuitry 160 determines values of patient parameters based on the signals. In some examples, sensors 166 determine the patient parameter values, and communicate them, e.g., via a wired or wireless connection, to processing circuitry 160.

In some examples, sensors 166 include one or more accelerometers 167, e.g., one or more 3-axis accelerometers. Signals generated by the one or more accelerometers 167 may be indicative of, as examples, gross body movement (e.g., activity) of patient 14, patient posture, heart sounds or other vibrations or movement associated with the beating of the heart, or coughing, rales, or other respiration abnormalities. In some examples, sensors 166 include one or more microphones configured to detect heart sounds or respiration abnormalities, and/or other sensors configured to detect patient activity or posture, such as gyroscopes and/or strain gauges. In some examples, sensors 166 may include sensors configured to transduce signals indicative of blood flow, oxygen saturation of blood, or patient temperature, and processing circuitry 160 may determine patient parameters values based on these signals.

In some examples, sensors 166 include one or more pressure sensors that transduce one or more signals indicative of blood pressure, and processing circuitry 160 determines one or more patient parameter values based on the pressure signals. Patient parameter values determined based on pressure may include, as examples, systolic or diastolic pressure values, such as pulmonary artery diastolic pressure values. In some examples, a separate pressure-sensing IMD 50 includes one or more sensors and sensing circuitry configured to generate a pressure signal, and processing circuitry 160 determines patient parameter values related to blood pressure based on information received from IMD 50.

Therapy delivery circuitry 164 is configured to generate and deliver electrical therapy to the heart. Therapy delivery circuitry 164 may include one or more pulse generators, capacitors, and/or other components capable of generating and/or storing energy to deliver as pacing therapy, defibrillation therapy, cardioversion therapy, other therapy or a combination of therapies. In some instances, therapy delivery circuitry 164 may include a first set of components configured to provide pacing therapy and a second set of components configured to provide anti-tachyarrhythmia shock therapy. In other instances, therapy delivery circuitry 164 may utilize the same set of components to provide both pacing and anti-tachyarrhythmia shock therapy. In still other instances, therapy delivery circuitry 164 may share some of the pacing and shock therapy components while using other components solely for pacing or shock delivery.

Therapy delivery circuitry 164 may include charging circuitry, one or more charge storage devices, such as one or more capacitors, and switching circuitry that controls when the capacitor(s) are discharged to electrodes 190 and the widths of pulses. Charging of capacitors to a programmed pulse amplitude and discharging of the capacitors for a programmed pulse width may be performed by therapy delivery circuitry 164 according to control signals received from processing circuitry 160, which are provided by processing circuitry 160 according to parameters stored in memory 170. Processing circuitry 160 controls therapy delivery circuitry 164 to deliver the generated therapy to the heart via one or more combinations of electrodes 190, e.g., according to parameters stored in memory 170. Therapy delivery circuitry 164 may include switch circuitry to select which of the available electrodes 190 are used to deliver the therapy, e.g., as controlled by processing circuitry 160.

In some examples, IMD 10 may be configured to determine whether a physiological response, or a change or trend in physiological responses over time, crosses a threshold. For example, processing circuitry 160 may monitor the health of a subject by measuring heart rate in response to a sit-to-stand transition, which is one example of an exertion event. Processing circuitry 160 may determine a mean, median, and/or standard deviation for the heart-rate responses to sit-to-stand transitions. Processing circuitry 160 may then determine that a change in the heart-rate responses over time exceeds a threshold, such as a current increase that exceeds or falls below a threshold or threshold range of absolute or percentage changes values. The threshold may be defined in terms of a long-term mean, median, and/or heart-rate response of one or more prior responses, e.g., during a baseline or other period preceding the current transition. Processing circuitry 160 may generate an alert based on the determination that the change crosses the threshold. Processing circuitry 160 may generate the alert by causing communication circuitry 168 to transmit a signal to an external device indicating that the change in responses exceeds the threshold.

As another example, IMD 10 may additionally or alternatively be configured to detect a plurality of walking events, such as walking twenty steps after standing still, not moving, or sitting, which are other examples of exertion events. IMD 10 may detect the walking events based on signals from accelerometer(s) 166. For each walking event, processing circuitry 160 may determine a respiration response based on a sensed signal from sensing circuitry 162. Processing circuitry 160 may determine that a change in the respiration response over time crosses a threshold. The change may be a gradual trend or a single outlier measurement.

As another example, IMD 10 may additionally or alternatively be configured to detect a plurality of lie-sit transitions, which are another example of an exertion event, based on one or more signals from one or more accelerometers 167. Processing circuitry 160 may determine the blood-pressure response to each lie-sit transition and determine whether a change over time crosses a threshold. Processing circuitry 160 may determine the threshold based on previous responses to lie-sit transitions, including the historical mean, median, and/or range of variability (e.g., standard deviation or variance) of the response.

According to the physiological-response monitoring techniques described herein, processing circuitry 160 determines values for each of one or more physiological parameters for the patient associated with the detection of an exertion event. The determined physiological parameter values are stored as physiological parameter values 174 in memory 170.

The plurality of physiological parameters may include one or more parameters determined based on the cardiac electrogram, such as one or more heart rate parameters or one or more measures of heart rate variability. Other patient parameters determined based on the cardiac electrogram include morphological features of the cardiac electrogram, such as QRS width or duration, QT interval length, T-wave amplitude, R-R interval length, an interval between a peak and the end of the T-wave, a ratio between the T-wave peak to end interval and the QT interval lengths, or T-wave alternan. The presence of T-wave alternan may be detected as a periodic (e.g., beat-to-beat) variation in the amplitude or morphology of the T-wave. A T-wave alternan patient parameter value 174 may be an indication of the presence, number, frequency, or duration (total, mean, or median) of T-wave alternan episodes. Other patient parameter values 174 based cardiac electrogram morphological interval lengths may be means or medians of a plurality of measurements.

The plurality of physiological parameters may additionally or alternatively include at least one parameter indicative of edema, and processing circuitry 160 may determine values 174 of such physiological parameters based on sensed thoracic impedance, as described above. In some examples, a physiological parameter value 174 may be a maximum, minimum, mean, or median thoracic impedance value.

The plurality of physiological parameters may additionally or alternatively include at least one patient parameter indicative of cardiovascular pressure, and processing circuitry 160 may determine values 174 of such physiological parameters based on generated pressure waveform, e.g., generated by a sensor 166 or pressure-sensing IMD 50, as described above. The physiological parameter values 174 may include a maximum, minimum, mean, and/or median of systolic pressure and/or diastolic pressure, e.g., pulmonary artery diastolic pressure.

The plurality of physiological parameters may additionally or alternatively include at least one patient parameter determined based on patient respiration, and processing circuitry 160 may determine values 174 of such parameters based on a generated signal that varies based on respiration as described above, such as a signal that varies based on thoracic impedance. The physiological parameter values 174 may include a maximum, minimum, mean, and/or median of respiration rate.

Processing circuitry 160 may additionally or alternatively determine values 174 of one or more physiological parameters based on a generated signal that varies based on sound or other vibrations, which may indicate heart sounds, coughing, or rales. Physiological parameter values may include morphological measurements of the Si and S2 heart sounds, the presence or frequency of occurrence of S3 and/or S4 heart sounds, or the presence, number, frequency, or duration (total, mean or media) of episodes or coughing or rales. Other physiological parameter values 174 that processing circuitry 160 may additionally or alternatively determine based on signals generated by sensors 166 include maximum, minimum, mean, or median values of blood flow, blood oxygen saturation, or temperature.

Processing circuitry 160 determines a difference metric 176 for each of the plurality physiological parameters. Processing circuitry 160 determines the difference metric 176 for each physiological parameter based on a difference between a two values 174 of the physiological parameter associated with detection of the exertion event. The two values may include a first value determined at or near, e.g., just prior to, the commencement of the exertion event, and a second value determined during the exertion event, or some predetermined period of time after or near the end of the exertion event. In this manner, difference metric 176 represents the response of the physiological parameter to the exertion event. In some examples, processing circuitry 160 determines the difference metric 176 for each of the patient parameters according to the equation, (difference metric)=(parameter value after exertion event)−(parameter value immediately before exertion event). Another possible equation for difference metric 176 calculates the percentage difference in the parameter value: (difference metric)=[(parameter value after exertion event)−(parameter value immediately before exertion event)] divided by (parameter value immediately before exertion event). The parameter value after the exertion event may be measured immediately at the end of the exertion event or a period of time after the end of the exertion event. The period of time may be five seconds, thirty seconds, several minutes, or any other period of time. Similarly, the parameter value before the exertion event may be measured immediately before the beginning of the exertion event, during the exertion event, or a period of time before the beginning of the exertion event.

Processing circuitry 160 compares the responses to the exertion event, e.g., difference metrics 176, for each of the one or more patient parameters to one or more threshold values, e.g., which may define a threshold range. If the difference metric crosses the threshold, e.g., is greater than or less than (or greater than or equal to or less than or equal to) the threshold, processing circuitry 160 generates an alert that the responses to exertion events over time have crossed a threshold. Thresholds 180 may include predetermined, e.g., programmable threshold values, or values that are variable. Thresholds 180 may be determined based on one or more prior difference metrics 176 determined in response to one or more prior exertion events, e.g., a median or mean of prior difference metrics, e.g., from a fixed baseline period and/or a recently-preceding trend period. Processing circuitry may determine a threshold or threshold range by multiplying a median or mean of prior difference metrics by a coefficient value (or adding to and/or subtracting from the mean or median a value) representative of the expected or average variability of the responses.

In some examples, processing circuitry 160 may additionally control therapy delivery circuitry 162, a pump included in IMD 10, or another implanted or external medical device to deliver or modify a therapy such as a pacing therapy, a neuromodulation therapy, or a therapeutic substance based on the score crossing a threshold. In some examples, a clinician may prescribe or deliver, or control another device to deliver to modify, such a therapy based on the alert generated by processing circuitry 160.

Communication circuitry 168 includes any suitable hardware, firmware, software or any combination thereof for communicating with another device, such as an external device 30 or another IMD or sensor. Under the control of processing circuitry 160, communication circuitry 168 may receive downlink telemetry from and send uplink telemetry to external device 30 or another device with the aid of an antenna, which may be internal and/or external. In some examples, communication circuitry 168 may communicate with a local external device, and processing circuitry 160 may communicate with a networked computing device via the local external device and a computer network, such as the Medtronic CareLink® Network developed by Medtronic, plc, of Dublin, Ireland.

A clinician or other user may retrieve data from IMD 10 using external device 30 or another local or networked computing device configured to communicate with processing circuitry 160 via communication circuitry 168. The clinician may also program parameters of IMD 10 using external device 30 or another local or networked computing device. In some examples, the clinician may select patient parameters used to determine if a physiological response to an exertion event crosses a threshold, select values for a coefficient used to determine threshold 180, and receive alerts that indicate that a physiological response to an exertion event crosses a threshold.

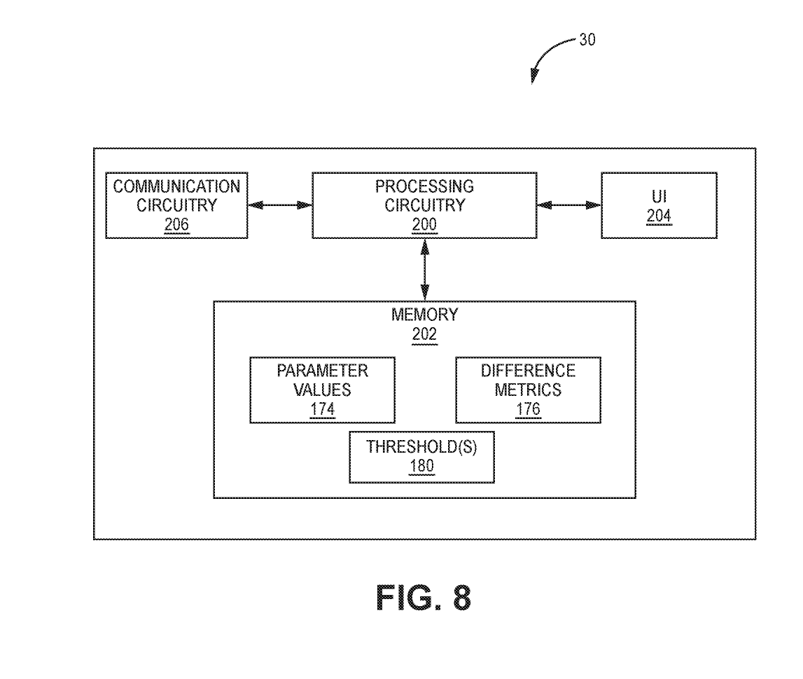

FIG. 8 is a functional block diagram illustrating an example configuration of an external device 30 configured to communicate with one or more IMDs 10. In the example of FIG. 8, external device 30 includes processing circuitry 200, memory 202, user interface (UI) 204, and communication circuitry 206. External device 30 may correspond to any of external devices 30A-30C described with respect to FIGS. 1, 2, and 4A-5. External device 30 may be a dedicated hardware device with dedicated software for the programming and/or interrogation of an IMD 10. Alternatively, external device 30 may be an off-the-shelf computing device, e.g., running an application that enables external device 30 to program and/or interrogate IMD 10.

In some examples, a user uses external device 30 to select or program any of the values for operational parameters of IMD 10, e.g., for patient parameter sensing, therapy delivery, and acute cardiac event prediction. In some examples, a user uses external device 30 to receive data collected by IMD 10, such as patient parameter values 174 or other operational and performance data of IMD 10. The user may also receive alerts generated by IMD 10 that indicate that a physiological response to an exertion event crosses a threshold. The user may interact with external device 30 via UI 204, which may include a display to present a graphical user interface to a user, and a keypad or another mechanism (such as a touch sensitive screen) for receiving input from a user. External device 30 may communicate wirelessly with IMD 10 using communication circuitry 206, which may be configured for RF communication with communication circuitry 168 of IMD 10.

Processing circuitry 200 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 200 may include multiple components, such as any combination of one or more microprocessors, one or more DSPs, one or more ASICs, or one or more FPGAs, as well as other discrete or integrated logic circuitry, and/or analog circuitry.

Memory 202 may store program instructions, which may include one or more program modules, which are executable by processing circuitry 200. When executed by processing circuitry 200, such program instructions may cause processing circuitry 200 and external device 30 to provide the functionality ascribed to them herein. The program instructions may be embodied in software, firmware and/or RAMware. Memory 202 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

In some examples, processing circuitry 200 of external device 30 may be configured to provide some or all of the functionality ascribed to processing circuitry 160 of IMD 10 herein. For example, processing circuitry 200 may receive physiological signals generated by one or more IMDs 10 and determine values 174 of each of a plurality of physiological parameters associated with exertion events, and/or may receive parameter values 174 from one or more IMDs 10. Processing circuitry 200 may determine difference metrics 176, and thresholds 180 based on the parameter values 174 in the manner described above with respect to processing circuitry 160 of IMD 10. Processing circuitry 200 may also compare difference metrics 176 to thresholds 180 and generate an alert and/or control delivery of therapy by one or more implanted or external medical devices in the manner described above with respect to processing circuitry 160 of IMD 10. Processing circuitry 200 may generate an alert to a user via UI 204, or via another device with which processing circuitry 200 communicates via communication circuitry 206.

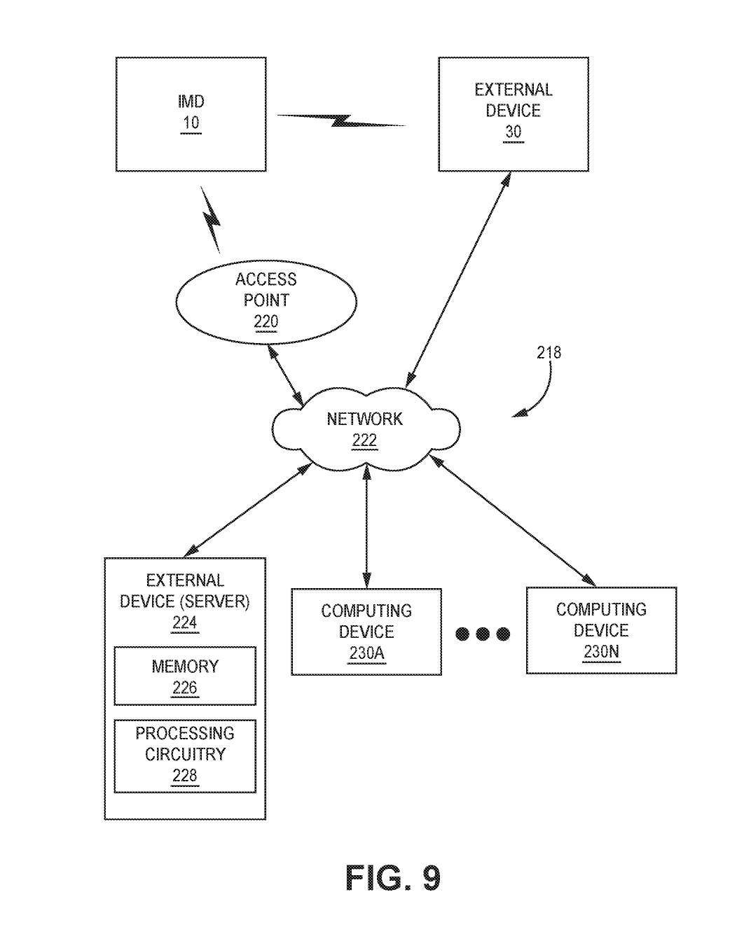

FIG. 9 is a functional block diagram illustrating an example system that includes external computing devices, such as a server 224 and one or more other computing devices 230A-230N, that are coupled to IMD 10 and external device 30 via a network 222. In this example, IMD 10 may use its communication module 168 to, e.g., at different times and/or in different locations or settings, communicate with external device 30 via a first wireless connection, and to communication with an access point 220 via a second wireless connection. In the example of FIG. 9, access point 220, external device 30, server 224, and computing devices 230A-230N are interconnected, and able to communicate with each other, through network 222.

Access point 220 may comprise a device that connects to network 222 via any of a variety of connections, such as telephone dial-up, digital subscriber line (DSL), or cable modem connections. In other examples, access point 220 may be coupled to network 222 through different forms of connections, including wired or wireless connections. In some examples, access point 220 may be co-located with patient 14. Access point 220 may interrogate IMD 10, e.g., periodically or in response to a command from patient 14 or network 222, to retrieve physiological signals, patient parameter values 174, difference metrics 176, scores 178, thresholds 180, alerts of crossed thresholds, and/or other operational or patient data from IMD 10. Access point 220 may provide the retrieved data to server 224 via network 222.

In some cases, server 224 may be configured to provide a secure storage site for data that has been collected from IMD 10 and/or external device 30. In some cases, server 224 may assemble data in web pages or other documents for viewing by trained professionals, such as clinicians, via computing devices 230A-230N. The illustrated system of FIG. 9 may be implemented, in some aspects, with general network technology and functionality similar to that provided by the Medtronic CareLink® Network developed by Medtronic plc, of Dublin, Ireland.

In some examples, one or more of access point 220, server 224, or computing devices 230 may be configured to perform, e.g., may include processing circuitry configured to perform, some or all of the techniques described herein, e.g., with respect to processing circuitry 160 of IMD 10 and processing circuitry 200 of external device 30, relating to physiological responses that cross a threshold. In the example of FIG. 9, server 224 includes a memory 226 to store physiological signals or patient parameter values 174 received from IMD 10 and/or external device 30, and processing circuitry 228, which may be configured to provide some or all of the functionality ascribed to processing circuitry 160 of IMD 10 and processing circuitry 200 of external device 30 herein. For example, processing circuitry 228 may determine values 174 of each of a plurality of physiological parameters, and/or may receive parameter values 174 from one or more IMDs 10. Processing circuitry 228 may determine difference metrics 176 and thresholds 180 based on the parameter values 174 in the manner described above with respect to processing circuitry 160 of IMD 10. Processing circuitry 227 may also compare difference metrics 176 to thresholds 180 and generate an alert and/or control delivery of preventative therapy by one or more implanted or external medical devices in the manner described above with respect to processing circuitry 160 of IMD 10. Processing circuitry 228 may generate an alert to a user via network 222, e.g., via external device 30 or one of computing devices 170.

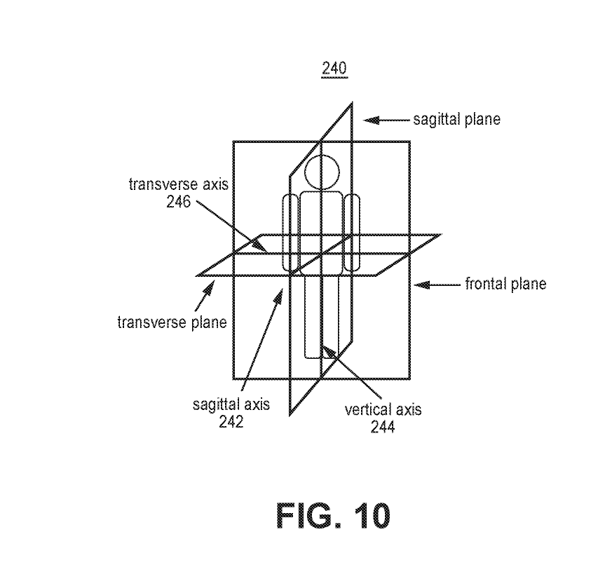

FIG. 10 is a conceptual diagram 240 illustrating a sagittal axis 242, a vertical axis 244 and transverse axis 246 in a three-dimensional coordinate system. As described above, an accelerometer 167 of an IMD 10 that is oriented along a depth (D) of the IMD may be oriented substantially along sagittal axis 242 when the IMD is implanted within a patient 14.

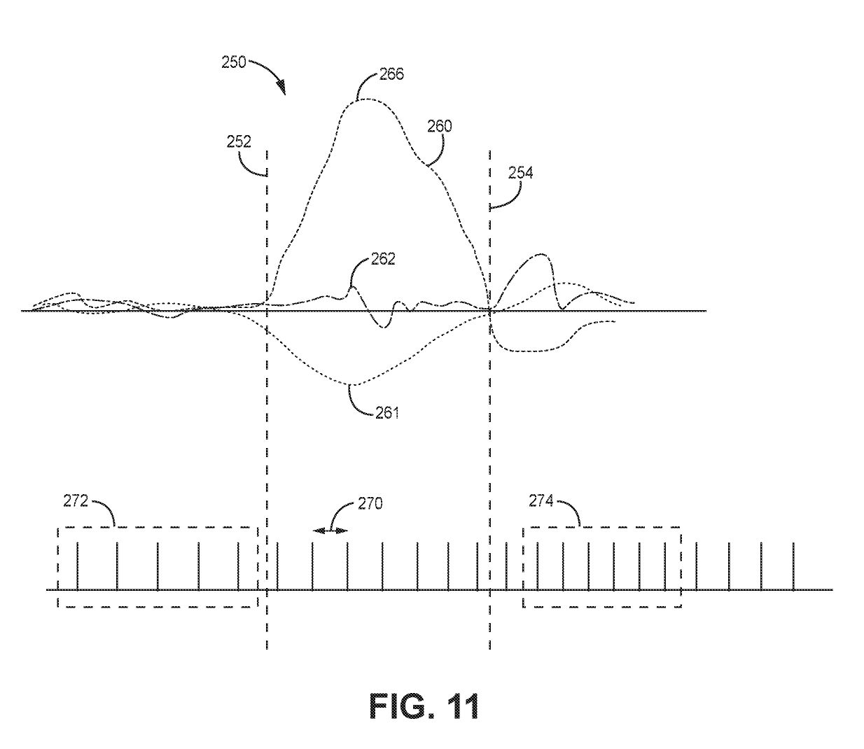

FIG. 11 is a timing diagram illustrating a chart of three accelerometer signals 260-262, where the three signals represent vertical acceleration, transverse acceleration, and sagittal acceleration. IMD 10 may comprise one or more accelerometers 167, e.g., one or more three-axis accelerometers. Signals generated by the one or more accelerometers, such as one or more of a sagittal axis signal, a vertical axis signal and a transverse axis signal, may be indicative of, as examples, gross body movement (e.g., activity) of a patient, patient posture, heart sounds or other vibrations or movement associated with the beating of the heart, or coughing, rales, or other respiration abnormalities. Three-axis accelerometers, as well as techniques for detecting sit-to-stand transitions or other posture transitions based on signals generated by three-axis accelerometers, are described in commonly-assigned U.S. Provisional Patent Application No. 62/370,138, titled, “ACCELEROMETER SIGNAL CHANGE AS A MEASURE OF PATIENT FUNCTIONAL STATUS,” bearing Attorney Docket Number C00012555.USP1, filed Aug. 2, 2016, the entire content of which is incorporated by reference herein.

Sagittal signal 260 may exhibit the largest amplitude swings during a posture transition 250 such as a sit-to-stand transition or a lay-sit transition. Transverse signal 261 may exhibit a negative amplitude swing during the posture transition 250, and vertical signal 262 may exhibit moderate variation during the posture transition 250. The posture transition may begin at approximately time 252 and end at approximately time 254. The peak 266 of the sagittal acceleration signal 260 during posture transition is also illustrated.

FIG. 11 also illustrates a marker channel chart showing detected heart beats (e.g., depolarizations) before, during, and after posture transition 250, which is an example of an exertion event. Intervals 270 (one of which is labeled for clarity) may indicate a time between beats, e.g., R-waves, and may be an R-R interval. Heart rate may be represented by or determined from, and is inversely proportional, to intervals 270. Shorter intervals 170 may indicate a faster heart rate, and longer intervals 170 may indicate a slower heart rate.

Time period 272 may be a time during which an IMD can measure the physiological parameter before its response to the exertion event. Time period 272 may occur before or during the exertion event. The IMD may continually store data indicating the physiological parameter in a memory buffer. When the IMD detects an exertion event, such as at approximately time 250, the IMD may move the data from time period 272 into data memory to preserve the data for comparison with data from time period 274. Time period 274 may be a time during which an IMD can measure the response of the physiological parameter to the exertion event. Time period 274 may occur after or during the exertion event.

FIG. 11 depicts the heart rate during time period 274 as faster than the rate during time period 272. An IMD and/or other device may determine the response to an exertion event, e.g., sit-to-stand transition or other postural transition, by determining the difference in heart rate between time period 274 and time period 272. An excessive or inadequate increase in heart rate in response to a postural transition may indicate declining patient health.

For example, when a person stands up, baroreceptor reflexes are rapidly activated to restore arterial pressure so that mean arterial pressure is not reduced by more than a few mmHg when a person is standing compared to lying down. However, in order to maintain this normal mean arterial pressure, the person who is standing upright has increased systemic vascular resistance (sympathetic mediated), decreased venous compliance (due to sympathetic activation of veins), decreased stroke volume (due to decreased preload), and increased heart rate (baroreceptor-mediated tachycardia). Patients with autonomic nerve dysfunction or hypovolemia will not be able to effectively utilize these compensatory mechanisms and therefore will display orthostatic hypotension. Consequently, such patients may have relatively lower changes in heart rate and higher decreases in pressure in response to posture transitions.

On the other hand, postural tachycardia syndrome (POTS) is an excessive increase in heart rate on assuming an upright posture from an initial sitting posture. POTS is associated with symptoms of orthostatic intolerance and sympathetic over-activity. POTS may be associated with brain hypoperfusion, usually in the absence of hypotension. POTS is described in Wieling et al., “Testing for Autonomic Neuropathy: Heart Rate Changes After Orthostatic Manoeuvres and Static Muscle Contractions,” Clinical Science (London), 64(6):581-6, 1983.

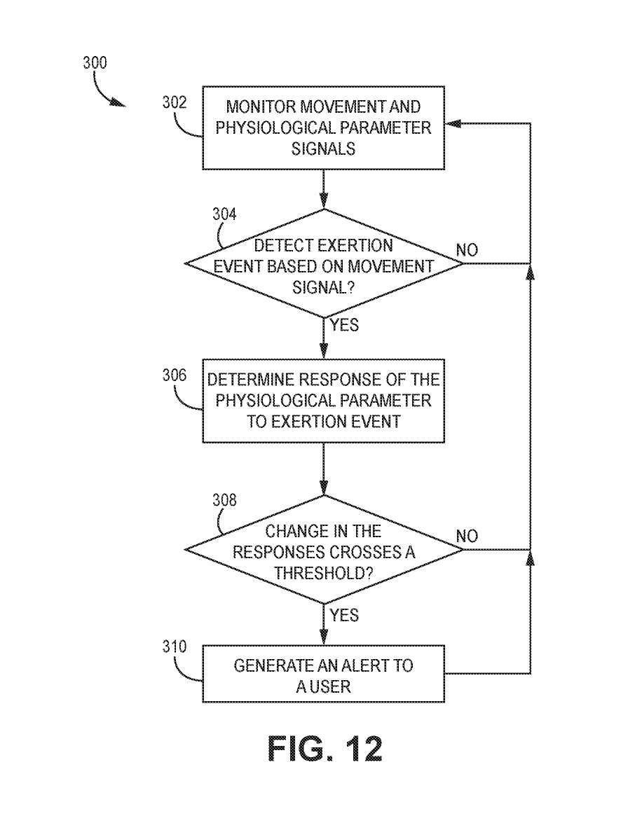

FIG. 12 is a flowchart illustrating an example technique 300 for determining whether a change in responses of a physiological parameter to an exertion event crosses a threshold, in accordance with this disclosure. Technique 300 may be implemented by any one of the implantable medical devices (IMDs) discussed above in connection with FIGS. 1-9, because each one of the IMDs is configured to include at least one accelerometer (i.e., accelerometer circuitry), as well as communication and processing circuitry (see FIG. 7 and corresponding description) to facilitate determining patient movements. More generally, technique may be performed, at least in part, by processing circuitry of any IMD and/or other device described herein.

The technique of FIG. 12 includes monitoring, e.g., by processing circuitry, a signal that varies as a function of movement of a subject as well as one or more other signals that vary as a function of a physiological parameter (302). The processing circuitry may monitor first sensed signals that vary as a function of movement of the subject using accelerometers. The first sensed signals may indicate exertion events such as posture transitions. The processing circuitry may monitor second sensed signals that vary as a function of a physiological parameter such as heart rate or blood pressure. The processing circuitry may monitor the second sensed signals through electrodes and/or sensing circuitry.

The technique of FIG. 12 further includes detecting an exertion event based on the signals (304). The processing circuitry may detect an exertion event by analyzing the patterns, amplitude, and duration of the first sensed signals. If the processing circuitry does not detect an exertion event, the processing circuitry may continue to monitor signals. In order to improve the accuracy of the measurements, technique 300 may require that the subject has not been physically active for the previous thirty minutes before the exertion event. Physical activity during the previous thirty minutes may substantially alter the response of the physiological parameter to the exertion event. Technique 300 may define “physical activity” by using the first sensed signals from the accelerometers.

If the processing circuitry detects an exertion event, the technique of FIG. 12 further includes determining the response of the physiological parameter to the exertion event (306). The physiological parameter, such as heart rate, blood pressure, or respiration, may increase in response to, e.g., during or immediately after, the exertion event. The response may comprise a difference or percentage difference in a measurement before the exertion event and a measurement after the exertion event. Technique 300 may comprise selecting a measurement before the exertion event with a minimum value and selecting a measurement during or after the exertion event with a maximum value.

The technique of FIG. 12 further includes determining whether a change in the responses over time crosses, e.g., exceeds and/or falls below, a threshold (308). The change in the responses over time may be a trend, or a difference and/or ratio between a measurement for a current exertion event and one or more prior measurements for one or more prior exertion events, e.g., a mean or median of prior measurements. The threshold may be a fixed value, or may vary over time. In some examples, the threshold may be determined based on one or more prior measurements, such that determining whether a change in response over time crosses a threshold comprises comparing a current response to a threshold determined based on prior responses.

If the IMD determines that a change in the responses crosses a threshold, the technique of FIG. 12 further includes generating an alert to a user (310). The alert may be an audible or visual alert, and may include an IMD transmitting a signal to an external device. The signal may indicate that the change in the responses crosses a threshold, along with pertinent details of the change in the responses.

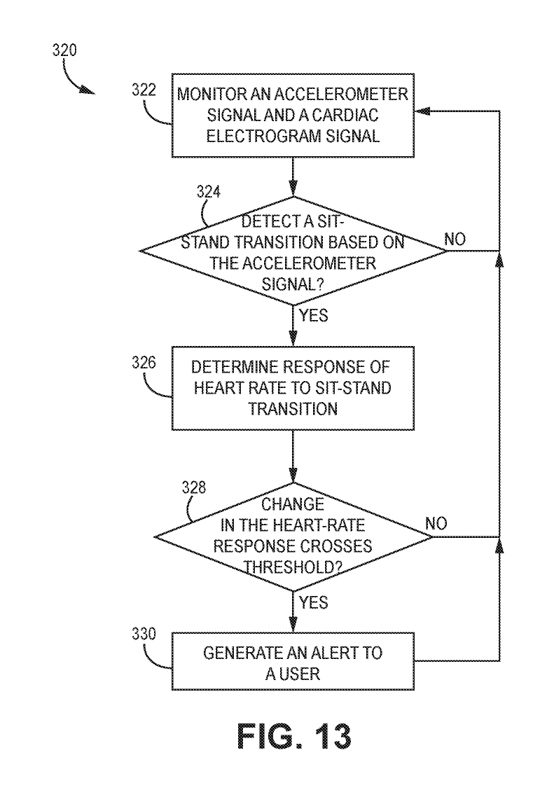

FIG. 13 is a flowchart illustrating an example technique 320 for determining whether a change in heart-beat responses to a sit-to-stand transition crosses a specific threshold, in accordance with this disclosure. Technique 320 may be a specific example of technique 300. Technique 320 may be implemented by any one of the implantable medical devices (IMDs) discussed above in connection with FIGS. 1-9, because each one of the IMDs is configured to include at least one accelerometer (i.e., accelerometer circuitry), as well as communication and processing circuitry (see FIG. 7 and corresponding description) to facilitate determining patient movements. More generally, technique may be performed, at least in part, by processing circuitry of any IMD and/or other device described herein.

The technique of FIG. 13 includes monitoring one or more accelerometer signals and one or more cardiac electrogram signals (322). The accelerometer signal may indicate the movement of the subject. The accelerometer signal may comprise three signals from an accelerometer, such as sagittal, vertical, and transverse. The cardiac electrogram signal may indicate the heart rate of the subject.

The technique of FIG. 13 further includes detecting a sit-to-stand transition based on the accelerometer signal (324). Processing circuitry may detect a sit-stand transition by analyzing the patterns, amplitude, and duration of the accelerometer signal. If the processing circuitry does not detect a sit-to-stand transition, the processing circuitry may continue to monitor the accelerometer signal.

In some examples, to detect sit-to-stand transitions, processing circuitry identifies a baseline of an accelerometer signal, e.g., sagittal signal 260. Identifying the baseline may include assigning a value of a current sample of the signal to value “0” by determining whether the current sample of the sagittal axis signal 1202 is within a certain number of units (e.g., 0.1 g) of baseline (e.g., 0 g) for at least a certain number (e.g., 15) of seconds. The processing circuitry also identifies the start 252 and end 254 of standing up by, for example, determining whether the amplitude of the accelerometer signal increases over a threshold (e.g. 0.2 g) and then decreases to less than the baseline within a certain time period (e.g. within 0.5 s-5 s). In some examples, processing circuitry also identifies a peak 266 of the accelerometer signal as a maximum value occurring between the start and end of the transition.

If the processing circuitry detects a sit-to-stand transition, the technique of FIG. 13 further includes determining the heart-rate response to the sit-to-stand transition (326). The heart rate of the subject may increase immediately after the sit-stand transition. The heart-rate response may comprise the difference between a heart rate during after the exertion event, e.g., a difference between a maximum heart rate during this period, and a heart rate before the exertion event.

The technique of FIG. 13 further includes determining whether a change in the heart-rate responses over time crosses a threshold (328). The change in the responses over time may be a trend, or a difference and/or ratio between heart rate response for a current sit-to-stand transition and one or more prior measurements for one or more prior exertion sit-to-stand transition, e.g., a mean or median of prior measurements. The threshold may be a fixed value, or may vary over time. In some examples, the threshold may be determined based on one or more prior measurements, such that determining whether a change in response over time crosses a threshold comprises comparing a current response to a threshold determined based on prior responses. For example, the threshold may be determined based on a mean or median of baseline or other prior sit-to-stand transition heart rate response measurements, modified by a value representative of an expected (predetermined and possibly programmed) or observed variability of the sit-to-stand transition heart rate response measurements.

In some examples, processing circuitry tracks median and variability (e.g., coefficient of variability (CV)) of sit-to-stand transition heart rate response measurements over time, and issues an alert of one of the sit-to-stand transition heart rate response measurements is an outlier. In some examples, the expected variability of sit-to-stand transition heart rate response measurements may be 15% to 40% increase in heart in response to the sit-to-stand transition. In some examples, the threshold range may have other percentage increase values, which may be determined based on observed variability. In some examples, a sit-to-stand transition heart rate change that is less than or falls below a lower threshold or threshold range bound, and/or that is greater than or exceeds an upper threshold or threshold range bound, is considered an outlier than crosses a threshold. The processing circuitry may generate an alert in response to the threshold being crossed.

If the IMD determines that the change in the heart-rate responses crosses the threshold, the technique of FIG. 13 further includes generating an alert to a user (330). The alert may be an audible or visual alert, or an IMD may transmit a signal to an external device. The signal may indicate that the change in the responses crosses a threshold, along with pertinent details of the change in the responses, such as the number of beats per minute by which each heart rate increase exceeded the threshold.

The flowcharts of FIGS. 12-13 are intended to illustrate the functional operation of an IMD 10, external device 30, medical system 8, and other devices and systems described herein, and should not be construed as reflective of a specific form of software or hardware necessary to practice the methods described. Methods described in conjunction with flow diagrams presented herein may be implemented in a non-transitory computer-readable medium that includes instructions for causing a programmable processor to carry out the methods described. A non-transitory computer-readable medium includes but is not limited to any volatile or non-volatile media, such as a RAM, ROM, CD-ROM, NVRAM, EEPROM, flash memory, or other computer-readable media, with the sole exception being a transitory, propagating signal. The instructions may be implemented by processing circuitry hardware as execution of one or more software modules, which may be executed by themselves or in combination with other software.

The example methods illustrated by FIGS. 12-13 may be performed, by any one or more devices described herein, and may be performed, in part, by processing circuitry of any one or more devices described herein, such as by processing circuitry 160 of IMD 10 (which may correspond to any of ICD 10A, ICM 10B, ICD 10C, IPD 10D, or any other IMD), processing circuitry 200 of external device 30, processing circuitry 228 of server 224.

Various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry, or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

### Exemplary Embodiments

Embodiment 1 is a method for monitoring health of a subject based on a physiological response to physical exertion comprising, by processing circuitry of a medical device system:

detecting a plurality of exertion events of the subject based on a first sensed signal that varies as a function of movement of the subject;

for each of the detected exertion events, determining a response of a physiological parameter of the subject to the exertion event based on second sensed signal that varies as a function of the physiological parameter;

determining a trend in the responses over time crosses a threshold; and

generating an alert to a user based on the determination that the trend crosses the threshold.

Embodiment 2 is the method of embodiment 1, further comprising determining the threshold based on at least one of a mean, a median, or a range of variability of the one or more previous responses of the physiological parameter to the exertion events.

Embodiment 3 is the method of embodiment 2, wherein the range of variability is based on a number of standard deviations from a mean or median of the one or more previous responses of the physiological parameter to the exertion events.

Embodiment 4 is the method of any of embodiments 1 to 3, wherein the first sensed signal comprises an accelerometer signal, and each of the exertion events comprises a common posture transition of the subject.

Embodiment 5 is the method of embodiment 4, wherein each of the posture transitions comprises a sit-stand transition.

Embodiment 6 is the method of any of embodiments 1 to 5, wherein the first sensed signal comprises an accelerometer signal, and each of the exertion events comprises a period of activity of the subject exceeding a threshold duration.

Embodiment 7 is the method of any one of embodiments 1 to 6, wherein the exertion event comprises a sit-stand transition, a stand-walk transition, or a walking after sitting.

Embodiment 8 is the method of any of embodiments 1 to 7, wherein the second sensed signal comprises a cardiac electrogram signal, and the physiological parameter comprises a heart rate.

Embodiment 9 is the method of any one of embodiments 1 to 8, wherein the physiological parameter comprises a blood pressure of the subject.

Embodiment 10 is the method of any one of embodiments 1 to 9, wherein generating the alert to the user comprises transmitting a signal to an external device indicating the trend in the responses over time crosses the threshold.

Embodiment 11 is a medical device system configured to monitor health of a subject based on a physiological response to physical exertion comprising:

sensing circuitry configured to:
generate a first sensed signal that varies as a function of movement of the subject; and
generate a second sensed signal that varies as a function of a physiological parameter of the subject;

processing circuitry configured to:
detect a plurality of exertion events of the subject based on the first sensed signal;
for each of the detected exertion events, determine a response of the physiological parameter of the subject to the exertion event based on the second sensed signal;
determine that a change in the responses over time crosses a threshold; and
generate an alert to a user in response to the determination that the change crosses the threshold.

Embodiment 12 is the medical device system of embodiment 11, wherein the first sensed signal comprises an accelerometer signal, and each of the exertion events comprises a common posture transition of the subject.

Embodiment 13 is the medical device system of embodiment 12, wherein each of the posture transitions comprises a sit-stand transition.

Embodiment 14 is the medical device system of any one of embodiments 11 to 13, wherein the first sensed signal comprises an accelerometer signal, and each of the exertion events comprises a period of activity of the subject exceeding a threshold duration.

Embodiment 15 is the medical device system of any one of embodiments 11 to 14, wherein the exertion event comprises a sit-stand transition, a stand-walk transition, or a walking after sitting.

Embodiment 16 is the medical device system of any one of embodiments 11 to 15, wherein the exertion event comprises standing after sitting, walking after standing still, or walking after sitting.

Embodiment 17 is the medical device system of any one of embodiments 11 to 16, wherein the second sensed signal comprises a cardiac electrogram signal, and the physiological parameter comprises a heart rate.

Embodiment 18 is the medical device system of any one of embodiments 11 to 17, wherein the physiological parameter comprises a blood pressure of the subject.

Embodiment 19 is the medical device system of any one of embodiments 11 to 18, wherein the processor is configured to generate the alert to the user by at least transmitting a signal to an external device indicating the change in the responses over time crosses the threshold.

Embodiment 20 is a non-transitory computer-readable storage medium comprising instructions, that when executed by processing circuitry of a medical device system, cause the medical device system to perform the method of any one of the above embodiments 1 to 10.

Embodiment 21 is a medical device system comprising means for performing the method of any one of embodiments 1 to 10.

Embodiment 22 is the method of any one of embodiments 1 to 10, wherein the response of the physiological parameter of the subject to the exertion event comprises a percentage change in the physiological parameter.

Embodiment 23 is the medical device system any one of embodiments 11 to 19, wherein the response of the physiological parameter of the subject to the exertion event comprises a percentage change in the physiological parameter.

Various aspects of this disclosure have been described. These and other aspects are within the scope of the following claims.

What is claimed is:

1. A method for monitoring health of a subject based on a physiological response to physical exertion comprising, by processing circuitry of a medical device system:
detecting a plurality of exertion events of the subject based on a first sensed signal that varies as a function of movement of the subject;
for each of the detected exertion events, determining a response of a physiological parameter of the subject to the exertion event based on second sensed signal that varies as a function of the physiological parameter;
determining that a change in the responses over time crosses a threshold; and
generating an alert to a user based on the determination that the change crosses the threshold.

2. The method of claim 1, further comprising determining the threshold for a current response of the physiological parameter to the exertion event based on one or more previous responses of the physiological parameter to the exertion event.

3. The method of claim 2, wherein determining the threshold comprises determining the threshold based on at least one of a mean, a median, or a range of variability of the one or more previous responses of the physiological parameter to the exertion events.

4. The method of claim 3, wherein the range of variability is based on a number of standard deviations from a mean or median of the one or more previous responses of the physiological parameter to the exertion events.

5. The method of claim 1, wherein each of the responses comprises a metric quantifying a change in the physiological parameter in response to a respective one of the exertion events.

6. The method of claim 1, wherein the first sensed signal comprises an accelerometer signal, and each of the exertion events comprises a common posture transition of the subject.

7. The method of claim 6, wherein each of the posture transitions comprises a sit-stand transition.

8. The method of claim 1, wherein the first sensed signal comprises an accelerometer signal, and each of the exertion events comprises a period of activity of the subject exceeding a threshold duration.

9. The method of claim 1, wherein the exertion event comprises a sit-stand transition, a stand-walk transition, or a walking after sitting.

10. The method of claim 1, wherein the second sensed signal comprises a cardiac electrogram signal, and the physiological parameter comprises a heart rate.

11. The method of claim 1, wherein the physiological parameter comprises a blood pressure of the subject.

12. The method of claim 1, wherein generating the alert to the user comprises transmitting a signal to an external device indicating the change in the responses over time crosses the threshold.

13. A medical device system configured to monitor health of a subject based on a physiological response to physical exertion comprising:
sensing circuitry configured to:
generate a first sensed signal that varies as a function of movement of the subject; and
generate a second sensed signal that varies as a function of a physiological parameter of the subject;
processing circuitry configured to:
detect a plurality of exertion events of the subject based on the first sensed signal;
for each of the detected exertion events, determine a response of the physiological parameter of the subject to the exertion event based on the second sensed signal;
determine that a change in the responses over time crosses a threshold; and
generate an alert to a user in response to the determination that the change crosses the threshold.

14. The medical device system of claim 13, further comprising a housing containing the sensing circuitry and the processing circuitry wherein the housing is configured for implantation in a human body.

15. The medical device system of claim 13, further comprising a memory configured to store the threshold and data indicating the change in responses over time the baseline change.

16. The medical device system of claim 13, wherein the processor is further configured to determine the threshold for a current response of the physiological parameter to the exertion event based on one or more previous responses of the physiological parameter to the exertion event.

17. The medical device system of claim 16, wherein the processor is configured to determine the threshold by at least determining the threshold based on at least one of a mean, a median, or a range of variability of the one or more previous responses of the physiological parameter to the exertion events.

18. The medical device system of claim 17, wherein the range of variability is based on a number of standard deviations from a mean or median of the one or more previous responses of the physiological parameter to the exertion events.

19. The medical device system claim 13, wherein each of the responses comprises a metric quantifying a change in the physiological parameter in response to a respective one of the exertion events.

20. The medical device system of claim 13, wherein the first sensed signal comprises an accelerometer signal, and each of the exertion events comprises a common posture transition of the subject.
